(19) United States
(12) Patent Application Publication
(10) Pub. No.: US 2025/0179535 A1
(43) Pub. Date: Jun. 5, 2025

(54) METHOD FOR CARRYING OUT THE COMBINED OPERATION OF A BIOETHANOL PRODUCTION UNIT AND A BIOGAS UNIT

(71) Applicant: Novozymes A/S, Bagsvaerd (DK)

(72) Inventors: Lindsey TUCKER, Fort Collins, CO (US); Kishore RANE, Raleigh, NC (US); Billy WHITLOCK, Lakewood, CO (US)

(73) Assignee: Novozymes A/S, Bagsvaerd (DK)

(21) Appl. No.: 18/840,008

(22) PCT Filed: Feb. 21, 2023
(86) PCT No.: PCT/US2023/062954

§ 371 Date: Aug. 20, 2024

Related U.S. Application Data
(60) Provisional application No. 63/312,383, filed on Feb. 21, 2022.

(51) Int. Cl. C12P 7/10; C12N 9/24

(52) U.S. Cl. CPC C12P 7/10; C12N 9/2402; C12P 2201/00

(57) ABSTRACT

The present invention concerns a method for carrying out the combined operation of a bioethanol production unit and a biogas unit, wherein a yield enhancing composition is added to the whole stillage that is fed to the mashing step or the biogas unit, the outflow of the biogas unit, the thin stillage that is fed to the mashing step, added to the residual materials resulting from purification of the corn oil or protein product that are fed to the biogas unit, the wet cake that is fed to the mashing step or the biogas unit; and/or biomass added to any one of the preceding steps.

The invention relates to a method for carrying out the combined operation of a bioethanol production unit and a biogas unit, wherein a yield enhancing composition comprising at least one, at least two, at least three, at least four, or at least five different types of enzymes and/or at least one, at least two, at least three, at least four, or at least five different microorganism strains is i) added to the whole stillage that is fed to the mashing step; and/or ii) added to the whole stillage that is fed to the biogas unit; and/or iii) added to the outflow of the biogas unit before being mashed with the milled grain (e.g., corn meal) in the mashing step; and/or iv) added to the thin stillage that is fed to the mashing step; and/or v) added to the residual materials resulting from purification of the corn oil that are fed to the biogas unit; and/or vi) added to the residual materials resulting from purification of the protein product that are fed to the biogas unit; and/or vii) added to the wet cake that is fed to the mashing step; and/or viii) added to the wet cake that is fed to the biogas unit; and/or ix) added to the biogas unit; and/or x) biomass added to any one of i)-ix).

### BACKGROUND

The production of ethanol from corn is known and is a technology which is widely used on an industrial scale, particularly in the USA. Prior art units are those with the following process steps:

Dry milling of corn with a screen size of 2-4 mm (Jacques, Lyons, and Kelsall, 2003), wherein a mass fraction of approximately 33 percent of the particles of the corn meal which is formed has a particle size of <0.59 mm (Jacques, Lyons, and Kelsall, 2003),

Mashing corn meal with process liquids which are primarily composed of thin stillage (10-50 percent of the process liquids (Jacques, Lyons, and Kelsall, 2003)) and condensate from animal feed evaporation. The dry matter content (TS content) of the mash is approximately 29-33 percent (Jacques, Lyons, and Kelsall, 2003). The TS content of the thin stillage is approximately 4.4-7.8 percent (Jacques, Lyons, and Kelsall, 2003). In this regard, approximately 0.01-0.08 t TS is used in the form of thin stillage per tonne of corn meal for mashing.

In a typical fermentation process, approximately 360 mg of nitrogen per L of mash is required for the yeast to grow (Jacques, Lyons, and Kelsall, 2003). Missing nitrogen is typically fed to the process as urea.

The mash is fed to a cooking stage. Here, the mash is typically heated to approximately 104-107 degrees centigrade (Walker, Abbas, Ingledew, and Pilgrim, 2017), then the temperature is reduced to approximately 85 degrees centigrade in a flash chamber and the starch is digested with the addition of enzymes. In this regard, a mash temperature of far above the gelatinization temperature of corn is set; this is 62-72 degrees centigrade for standard corn or 67-80 degrees centigrade for corn with a high amylose content (Walker, Abbas, Ingledew, and Pilgrim, 2017).

The mash from the cooking stage is fed to a fermentation step and the digested starch is converted by the yeasts into ethanol at between 25-35 degrees centigrade (Walker, Abbas, Ingledew, and Pilgrim, 2017). The mass fraction of unconverted starch, the residual starch content, is less than 1.5 percent.

Approximately 4 percent of the sugar and starch in the corn meal (Medina et al., 2009) is transformed into the unwanted by-product glycerin instead of ethanol; this corresponds to approximately 24 kg of glycerin per tonne of corn meal.

The ethanol-containing mash from the fermentation step is fed to a distillation step at approximately 108 degrees centigrade (Walker, Abbas, Ingledew, and Pilgrim, 2017) and the ethanol is removed from the mash. The ethanol-depleted mash formed thereby is known as whole stillage.

The entirety of the whole stillage is fed to a solid-liquid separation step, usually decanter centrifuges. The TS content of the solid phase, hereinafter termed wet cake, is approximately 29-39 percent (Walker, Abbas, Ingledew, and Pilgrim, 2017) and the TS content of the liquid phase, hereinafter termed the thin stillage, is approximately 4.4-7.8 percent (Jacques, Lyons, and Kelsall, 2003).

A portion of the thin stillage is used for mashing the corn meal. The remainder is evaporated to form what is known as syrup and the condensate is used as a process liquid for mashing. Corn oil can be separated from the syrup and can be recovered separately.

Syrup and wet cake are used as animal feed. Syrup and wet cake are often dried, but can also be used moist as animal feed. In total, approximately 0.25 t dry matter of animal feed per tonne of corn meal is typically produced.

This standard process suffers from the following disadvantages:
The cooking stage and distillation step are carried out at high temperatures, which leads to a high heat energy consumption.
Solid-liquid separation of the entire amount of generated whole stillage leads to a high electrical energy consumption.
The thin stillage is evaporated, to obtain process liquids, which leads to a high heat energy consumption.
Typical further drying processes for the syrup and wet cake bring about a high heat energy consumption, but are necessary in typical configurations for the units in order to obtain a more attractive animal feed.
Nitrogen has to be added in the form of urea or similar chemicals.
The energy and chemical consumptions mentioned have a negative effect on the greenhouse gas balance for the fuels which are produced. An example of the quantification of the negative effects is the carbon intensity score (CI score) from the California Low-Carbon Fuel Standard. The CI score is a measure of the greenhouse gas emissions of fuels in grams of carbon dioxide equivalents (gCO₂e). A typical ethanol unit in the USA produces fuels with a CI score of approximately 79 gCO2e/MJ. The production process in the ethanol unit makes approximately 32 gCO₂e/MJ¹ of this, the remainder is from other emissions in the process line such as, for example, corn cultivation.
A typical ethanol unit has a high water consumption of approximately 2.7 L of fresh water per L of ethanol (Mueller, 2010), which corresponds to approximately 1.2 m³ per tonne of corn meal.

Cooking stages at temperatures below the gelatinization temperature of starches, what are known as cold mash processes, are known and bring about a lower heat energy consumption, but lead to losses which are too high because of residual starches and the risk of bacterial contamination. Comminution of the particle size of the corn meal, changing the pH and greater use of enzymes can reduce these losses and disadvantages (Walker, Abbas, Ingledew, and Pilgrim, 2017). These known measures alone, however, have not been enough in the past, and so the cold mash process has been replaced by the hot mash process.

Furthermore, processes are known which reduce the energy consumption for animal feed production by processing stillage into biogas instead of into animal feed. EP 2 501 818 B1 describes a process in which the stillage from an ethanol unit is fed to a biogas unit and a portion of the outflow from the biogas unit is recycled directly to the ethanol unit. U.S. Pat. No. 8,962,309 B2 describes a biogas unit with different types of biogas fermenters for thin stillage and wet cake. The biogas fermenters therein should be operated at an ammonium concentration of less than 6000 ppm NH4-N. WO 2013/000925 A1 describes a process in which thin stillage is fed to a biogas fermenter of the continuous stirred tank reactor (CSTR) type with a mean hydraulic residence time of 1-20 days. US2022/0033860 describes a method for carrying out the combined operation of a cold cook ethanol production unit and a biogas unit, wherein cellulases or cellulase-forming microorganisms are added to the outflow of a biogas unit that is then recycled to the mashing step.

### SUMMARY OF THE INVENTION

The problem to be solved by the invention is to operate a combination of a bioethanol unit and a biogas unit which saves energy, water and chemicals compared with a typical ethanol unit as described above and which, despite the energy savings, has high ethanol yields per tonne of milled grain (e.g., corn meal) and enhanced ethanol yields and/or biogas yields. In addition, the use of outflow from the biogas unit as process liquids for the ethanol unit should not have a negative effect on the ethanol fermentation.

The present invention engages with the problem set out above and solves it by providing a method for carrying out the combined operation of a bioethanol production unit and a biogas unit, comprising:
a) mashing milled grain from a dry milling step with at least 0.1 t of dry matter in the form of whole stillage and at least 0.1 m³ of outflow from the biogas unit per tonne of milled grain,
b) feeding the mash from a) to a cooking stage with mash temperatures below the gelatinization temperature of the starch in the milled grain, followed by an ethanol-forming fermentation step and then feeding the fermented mash to a distillation step to produce whole stillage,
c) feeding the whole stillage from b) to the mashing step in a) and to the biogas unit,
   wherein a yield enhancing composition comprising at least one, at least two, at least three, at least four, or at least five different types of enzymes and/or at least one, at least two, at least three, or at least four different microbial strains is:
i) added to the whole stillage from b) that is fed to the mashing step in a); and/or
ii) added to the whole stillage from b) that is fed to the biogas unit; and/or
iii) added to the outflow of the biogas unit before being mashed with the milled grain from step 1a); and/or
iv) added to the thin stillage that is fed to the mashing step; and/or
v) added to the residual materials resulting from purification of the corn oil that are fed to the biogas unit; and/or
vi) added to the residual materials resulting from purification of the protein product that are fed to the biogas unit; and/or
vii) added to the wet cake that is fed to the mashing step; and/or
viii) added to the wet cake that is fed to the biogas unit; and/or
ix) added to the biogas unit; and/or
x) biomass added to any one of i)-ix).

In step a), milled grain, preferably corn meal, from a grain, preferably corn, dry milling step is mashed with liquids. These liquids consist of at least 0.1 t TS of whole stillage and at least 0.1 m³ of outflow from the biogas unit per tonne of milled grain (e.g., corn meal).

The recycle of outflow from the biogas unit with the yield enhancing composition has the advantage that ethanol yields can be enhanced by providing enzymes and/or microorganisms that can degrade materials in the outflow remaining post anerobic digestion, such as residual starch, cellulose, hemicellulose, yeast cell wall beta-glucan, protein, etc. to produce additional fermentable sugars and nitrogen to fuel the yeast during fermentation, while savings can also be made as regards fresh water as well as process liquids. In a typical ethanol unit, process liquids are obtained in an energy-intensive manner by the evaporation of thin stillage.

In step b), the mash from step a) is fed to a cooking stage in which the mash is heated to temperatures below the gelatinization temperature of the starch in the milled grain. This results in significant savings in energy compared with typical cooking stages at 104-107 degrees centigrade. The mash from the cooking stage is fed to a fermentation step in which ethanol is formed. The ethanol-containing mash from the fermentation step is then fed to a distillation step in which the ethanol is separated out.

In step c), a portion of the ethanol-depleted mash from the distillation step, what is known as the whole stillage, is fed without the typical solid-liquid separation directly to the mashing step a) and to the biogas unit. Compared with typical solid-liquid separation of the whole stillage in its entirety, this means that savings are made as regards the electrical energy for the solid-liquid separation of whole stillage and a large proportion of the residual starch from the fermentation step is recycled which would otherwise no longer be available for ethanol formation because it would have been used as animal feed. The addition of a yield enhancing composition comprising at least one, at least two, at least three, at least four, or at least five different types of enzymes and/or at least one, at least two, at least three, or at least four different microbial strains formulated to enhance ethanol yield and/or biogas yield is i) added to the whole stillage from b) that is fed to the mashing step in a); and/or ii) added to the whole stillage from b) that is fed to the biogas unit; and/or iii) added to the outflow of the biogas unit before being mashed with the milled grain from step 1a) and/or iv) added to the thin stillage that is fed to the mashing step; and/or v) added to the residual materials resulting from purification of the corn oil that are fed to the biogas unit; and/or vi) added to the residual materials resulting from purification of the protein product that are fed to the biogas unit; and/or vii) added to the wet cake that is fed to the mashing step; and/or viii) added to the wet cake that is fed to the biogas unit; and/or ix) added to the biogas unit; and/or x) biomass added to any one of i)-ix).

The addition of the yield enhancing composition in this manner further increases ethanol yield, biogas yield, and/or ethanol yield and biogas yield.

### BRIEF DESCRIPTION OF THE FIGURES

The invention will now be described in more detail with the aid of two exemplary embodiments and associated drawings, in which:

FIG. 1 and FIG. 2 diagrammatically describe the process workflow for embodiments in accordance with the invention.

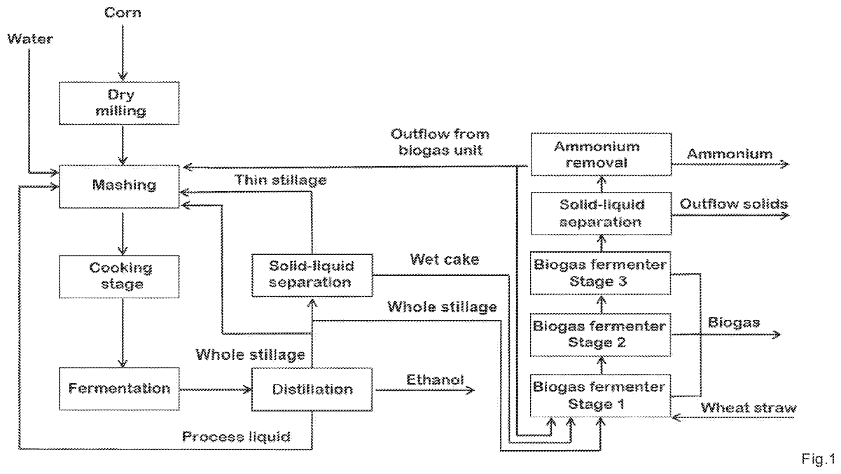

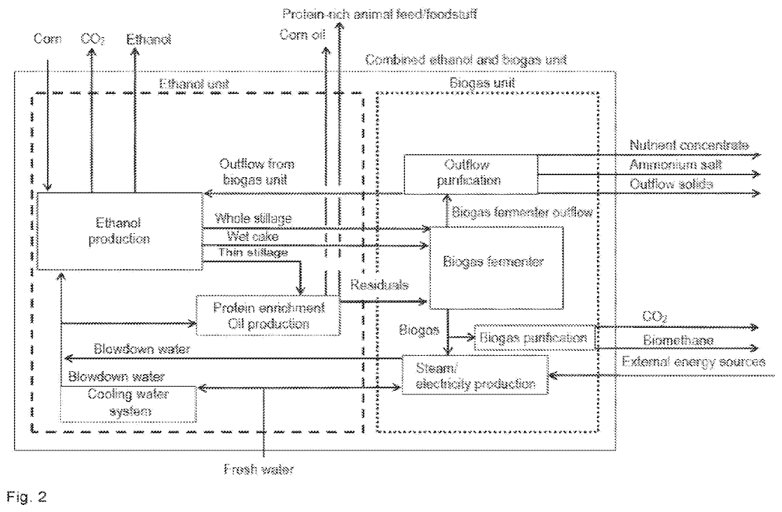

### DETAILED DESCRIPTION OF THE INVENTION

### Definitions

The term “dry matter” (TS) means the solid residue which is obtained after removing the solvent (for example water or ethanol) from a suspension (for example from stillage) or from a solution. The solid residue means all of the previously dissolved or suspended solids (for example raw proteins, yeast and salts). The mass of the dry matter is known as the dry mass and can be given in kilograms. The term “dry matter content” (TS content) means the percentage mass fraction of the dry matter with respect to the total mass of the suspension (for example the stillage) or solution.

The term “stillage” means the residue from distillation of an ethanol-containing grain mash. The term “whole stillage” is synonymous with “stillage”.

The term “solid-liquid separation” means a process which separates a suspension (for example stillage) into a two-phase system comprising a solid phase and a liquid phase. The solid-liquid separation step may advantageously be carried out in a separator or decanter. The term “solid phase” should be understood, in a two-phase system, to mean the phase which has the higher dry matter content. A solid phase in this regard may include a suspension or a sedimented solid (residue). The term “liquid phase” in a two-phase system means the phase which has the lower dry matter content. In this regard, a liquid phase may include a suspension or a clear solution.

The term “thin stillage” means a liquid phase produced by solid-liquid separation of whole stillage. The TS content of a thin stillage can advantageously be at least 8 percent. The term “wet cake” as used in the context of this invention is defined as the solid phase which is separated from the stillage by solid-liquid separation.

The term “outflow solid” means the solid phase which is separated from the outflow from a biogas fermenter by solid-liquid separation.

The term “ammonium nitrogen content” (NH4-N content) means the mass fraction of nitrogen in the form of ammonium in a sample. In order to measure the NH4-N content of a sample, the sample is made basic with sodium hydroxide and then distilled with steam. Ammonia is driven out of the sample and trapped in a boric acid receiving tank. The mass fraction of ammonium nitrogen is determined by titration of the borate, which is formed, using dilute hydrochloric acid.

The terms “biomass” as used herein includes any polymeric sugar containing material. Preferably, biomass as used herein includes cellulosic material and/or lignocellulosic material and/or hemicellulosic material and/or starch material and/or pectin material and/or lipid material. Biomass suitable for use in the processes as described herein includes virgin biomass and/or non-virgin biomass such as agricultural biomass, processed agricultural biomass, commercial organics, construction and demolition debris, municipal solid waste, household waste, waste paper and yard waste, waste of the dairy industry (comprising for example lactose), waste from the beet and cane sugar industry (comprising for example sucrose (saccharose)) and by-products of oil seeds (for example rape seed meal, sunflower meal and soy husks). Common forms of biomass include trees, shrubs and grasses, wheat, rye, oat, wheat straw, sugar cane, cane straw, sugar cane bagasse, switch grass, miscanthus, energy cane, cassava, molasse, barley, corn, corn stover, corn fiber, corn husks, corn cobs, canola stems, soybean stems, sweet sorghum, corn kernel including fiber from kernels, distillers dried grains (DDGS), brewery spent grains, silage such as corn silage, grass silage and beet silage, products and by-products from milling of grains such as corn, wheat and barley (including wet milling and dry milling) often called “bran or fibre” as well as municipal solid waste, waste paper and yard waste. The biomass can also be, but is not limited to, herbaceous material, agricultural residues, forestry residues, municipal solid wastes, waste woods (type A, B and/or C), waste paper, and pulp and paper mill residues. “Agricultural biomass” includes branches, bushes, canes, corn and corn husks, energy crops, forests, fruits, flowers, grains, grasses, herbaceous crops, leaves, bark, needles, logs, roots, saplings, short rotation woody crops, shrubs, switch grasses, trees, vegetables, fruit peels, vines, sugar beet, sugar beet pulp, wheat midlings, oat hulls, and hard and soft woods (not including woods with deleterious materials). In addition, agricultural biomass includes organic waste materials generated from agricultural processes including farming and forestry activities, specifically including forestry wood waste. Agricultural biomass may be any of the afore-mentioned singularly or in any combination or mixture thereof. In a preferred embodiment the biomass is silage.

A “different type of enzyme” means that the enzyme catalyzes a different reaction even though the enzyme may be within the same class. For instance, endoglucanase and cellobiohydrolase catalyze different reactions though they are both cellulases, accordingly endoguclanase and cellobiohydrolase are considered a different type of enzyme according to the instant disclosure.

A “different microorganism strain” means that the strain is from a different genus or species of microorganism, though the microorganism may be the same type of microorganism. For instance, Bacillus subtillis and Bacillus amyloliquefaciens strains are different microorganism even though they are both bacterial strains.

A “yield enhancing composition” is a composition that increases yield by at least 1% within a margin of error of 0.2%. The yield enhancing composition may enhance yield of any product, intermediate product, or co-product described herein. The yield enhancing composition may enhance ethanol yield, biogas yield, protein yield, oil yield, etc.

An a-L-arabinofuranosidase (EC 3.2.1.55) is any polypeptide which is capable of acting on a-L-arabinofuranosides, a-L-arabinans containing (1,2) and/or (1,3)- and/or (1,5)-linkages, arabinoxylans and arabinogalactans. This enzyme may also be referred to as a-N-arabinofuranosidase, arabinofuranosidase or arabinosidase.

An acetyl xylan esterase (EC 3.1.1.72) is any polypeptide which is capable of catalysing the deacetylation of xylans and xylo-oligosaccharides. Such a polypeptide may catalyze the hydrolysis of acetyl groups from polymeric xylan, acetylated xylose, acetylated glucose, alpha-napthyl acetate or p-nitrophenyl acetate but, typically, not from triacetylglycerol. Such a polypeptide typically does not act on acetylated mannan or pectin.

“Amylase” means enzymes that hydrolyze alpha-1,4-glucosidic linkages in starch, both in amylose and amylopectin, such as alpha-amylase (EC 3.2.1.1), beta-amylase (EC 3.2.1.2), glucan 1,4-alpha-glucosidase (EC 3.2.1.3), glucan 1,4-alpha-maltotetraohydrolase (EC 3.2.1.60), glucan 1,4-alpha-maltohexaosidase (EC 3.2.1.98), glucan 1,4-alpha-maltotriohydrolase (EC 3.2.1.116) and glucan 1,4-alpha-maltohydrolase (EC 3.2.1.133), and enzymes that hydrolyze alpha-1,6-glucosidic linkages, being the branch-points in amylopectin, such as pullulanase (EC 3.2.1.41) and limit dextinase (EC 3.2.1.142).

Expansins are implicated in loosening of the cell wall structure during plant cell growth. Expansins have been proposed to disrupt hydrogen bonding between cellulose and other cell wall polysaccharides without having hydrolytic activity. In this way, they are thought to allow the sliding of cellulose fibers and enlargement of the cell wall. Swollenin, an expansin-like protein contains an N-terminal Carbohydrate Binding Module Family 1 domain (CBD) and a C-terminal expansin-like domain. As described herein, an expansin-like protein orswollenin-like protein may comprise one or both of such domains and/or may disrupt the structure of cell walls (such as disrupting cellulose structure), optionally without producing detectable amounts of reducing sugars. A cellulose induced protein, for example the polypeptide product of the cip1 or cip2 gene or similar genes (see Foreman et al., J. Biol. Chem. 278 (34), 31988-31997, 2003), a cellulose/cellulosome integrating protein, for example the polypeptide product of the cipA or cipC gene, or a scaffoldin or a scaffoldin-like protein. Scaffoldins and cellulose integrating proteins are multi-functional integrating subunits which may organize cellulolytic subunits into a multi-enzyme complex. This is accomplished by the interaction of two complementary classes of domain, i.e. a cohesion domain on scaffoldin and a dockerin domain on each enzymatic unit. The scaffoldin subunit also bears a cellulose-binding module (CBM) that mediates attachment of the cellulosome to its substrate. A scaffoldin or cellulose integrating protein may comprise one or both of such domains.

“Glucuronidase” includes enzymes that catalyze the hydrolysis of a glucuronoside, for example b-glucuronoside to yield an alcohol. Many glucuronidases have been characterized and may be suitable for use, for example b-glucuronidase (EC 3.2.1.31), hyalurono-glucuronidase (EC 3.2.1.36), glucuronosyl-disulfoglucosamine glucuronidase (3.2.1.56), glycyrrhizinate b-glucuronidase (3.2.1.128) or a-D-glucuronidase (EC 3.2.1.139).

An “a-D-glucuronidase” (EC 3.2.1.139) is any polypeptide which is capable of catalyzing a reaction of the following form: alpha-D-glucuronoside+H(2)0=an alcohol+D-glucuronate. This enzyme may also be referred to as alpha-glucuronidase or alpha-glucosiduronase. These enzymes may also hydrolyze 4-O-methylated glucuronic acid, which can also be present as a substituent in xylans. An alternative is EC 3.2.1.131: xylan alpha-1,2-glucuronosidase, which catalyses the hydrolysis of alpha-1,2-(4-0-methyl)glucuronosyl links.

A beta-(1,3)(1,4)-glucanase (EC 3.2.1.73) is any polypeptide which is capable of catalyzing the hydrolysis of 1,4-p-D-glucosidic linkages in b-D-glucans containing 1,3- and 1,4-bonds. Such a polypeptide may act on lichenin and cereal b-D-glucans, but not on b-D-glucans containing only 1,3- or 1,4-bonds. This enzyme may also be referred to as licheninase, 1,3-1,4-b-D-glucan 4-glucanohydrolase, b-glucanase, endo-b-1,3-1,4 glucanase, lichenase or mixed linkage b-glucanase. An alternative for this type of enzyme is EC 3.2.1.6, which is described as endo-1,3 (4)-beta-glucanase. This type of enzyme hydrolyses 1,3- or 1,4-linkages in beta-D-glucanse when the glucose residue whose reducing group is involved in the linkage to be hydrolysed is itself substituted at C-3. Alternative names include endo-1,3-beta-glucanase, laminarinase, 1,3-(1,3; 1,4)-beta-D-glucan 3 (4) glucanohydrolase. Substrates include laminarin, lichenin and cereal beta-D-glucans.

A coumaroyl esterase (EC 3.1.1.73) is any polypeptide which is capable of catalyzing a reaction of the form: coumaroyl-saccharide+H(2)0=coumarate+saccharide. The saccharide may be, for example, an oligosaccharide or a polysaccharide. This enzyme may also be referred to as trans-4-coumaroyl esterase, trans-p-coumaroyl esterase, p-coumaroyl esterase or p-coumaric acid esterase. This enzyme also falls within EC 3.1.1.73 so may also be referred to as a feruloyl esterase.

“Catalase” means a hydrogen-peroxide: hydrogen-peroxide oxidoreductase (EC 1.11.1.6 or EC 1.11.1.21) that catalyzes the conversion of two hydrogen peroxides to oxygen and two waters. Catalase activity can be determined by monitoring the degradation of hydrogen peroxide at 240 nm based on the following reaction: 2H2O2 (R) 2H2O+O2. The reaction is conducted in 50 mM phosphate pH 7.0 at 25 degrees centigrade with 10.3 mM substrate (H2O2) and approximately 100 units of enzyme per ml. Absorbance is monitored spectrophotometrically within 16-24 seconds, which should correspond to an absorbance reduction from 0.45 to 0.4. One catalase activity unit can be expressed as one micromole of H2O2 degraded per minute at pH 7.0 and 25 degrees centigrade.

A “cellulase” is any polypeptide which is capable of degrading or modifying cellulose. A polypeptide which is capable of degrading cellulose is one which is capable of catalyzing the process of breaking down cellulose into smaller units, either partially, for example into cellodextrins, or completely into glucose monomers. A cellulase as described herein may give rise to a mixed population of cellodextrins and glucose monomers. Such degradation will typically take place by way of a hydrolysis reaction.

“Endoglucanases” are enzymes which are capable of catalyzing the endohydrolysis of 1,4-p-D-glucosidic linkages in cellulose, lichenin or cereal b-D-glucans. They belong to EC 3.2.1.4 and may also be capable of hydrolyzing 1,4-linkages in b-D-glucans also containing 1,3-linkages. Endoglucanases may also be referred to as cellulases, avicelases, b-1,4-endoglucan hydrolases, b-1,4-glucanases, carboxymethyl cellulases, celludextrinases, endo-1,4-b-D-glucanases, endo-1,4-D-glucanohydrolases or endo-1,4-glucanases.

A “beta-glucosidase” (EC 3.2.1.21) is any polypeptide which is capable of catalyzing the hydrolysis of terminal, non-reducing b-D-glucose residues with release of b-D-glucose. Such a polypeptide may have a wide specificity for b-D-glucosides and may also hydrolyze one or more of the following: a b-D-galactoside, an a-L-arabinoside, a b-D-xyloside or a b-D-fucoside. This enzyme may also be referred to as amygdalase, b-D-glucoside glucohydrolase, cellobiase or gentobiase.

A “cellobiohydrolase” (EC 3.2.1.91) is any polypeptide which is capable of catalyzing the hydrolysis of 1,4-D-glucosidic linkages in cellulose or cellotetraose, releasing cellobiose from the ends of the chains. This enzyme may also be referred to as cellulase 1,4-b-cellobiosidase, 1,4-cellobiohydrolase, 1,4-D-glucan cellobiohydrolase, avicelase, bco-1,4-b-0-glucanase, exocellobiohydrolase or exoglucanase.

A “hemicellulase” is any polypeptide which is capable of degrading or modifying hemicellulose. That is to say, a hemicellulase may be capable of degrading or modifying one or more of xylan, glucuronoxylan, arabinoxylan, glucomannan and xyloglucan. A polypeptide which is capable of degrading a hemicellulose is one which is capable of catalyzing the process of breaking down the hemicellulose into smaller polysaccharides, either partially, for example into oligosaccharides, or completely into sugar monomers, for example hexose or pentose sugar monomers. A hemicellulase as described herein may give rise to a mixed population of oligosaccharides and sugar monomers. Such degradation will typically take place by way of a hydrolysis reaction.

An “a-L-arabinofuranosidase” (EC 3.2.1.55) is any polypeptide which is capable of acting on a-L-arabinofuranosides, a-L-arabinans containing (1,2) and/or (1,3)- and/or (1,5)-linkages, arabinoxylans and arabinogalactans. This enzyme may also be referred to as a-N-arabinofuranosidase, arabinofuranosidase or arabinosidase.

An “endo-arabinanase” (EC 3.2.1.99) is any polypeptide which is capable of catalysing endohydrolysis of 1,5-a-arabinofuranosidic linkages in 1,5-arabinans. The enzyme may also be known as endo-arabinase, arabinan endo-1,5-a-L-arabinosidase, endo-1,5-a-L-arabinanase, endo-α-1,5-arabanase; endo-arabanase or 1,5-a-L-arabinan 1,5-a-L-arabinanohydrolase.

An “endoxylanase” (EC 3.2.1.8) is any polypeptide which is capable of catalysing the endohydrolysis of 1,4-p-D-xylosidic linkages in xylans. This enzyme may also be referred to as endo-1,4-p-xylanase or 1,4-p-D-xylan xylanohydrolase. An alternative is EC 3.2.1.136, a glucuronoarabinoxylan endoxylanase, an enzyme that is able to hydrolyze 1,4 xylosidic linkages in glucuronoarabinoxylans.

A “feruloyl esterase (EC 3.1.1.73)” is any polypeptide which is capable of catalyzing a reaction of the form: feruloyl-saccharide+H2O=ferulate+saccharide. The saccharide may be, for example, an oligosaccharide or a polysaccharide. It may typically catalyze the hydrolysis of the 4-hydroxy-3-methoxycinnamoyl (feruloyl) group from an esterified sugar, which is usually arabinose in ‘natural’ substrates p-nitrophenol acetate and methyl ferulate are typically poorer substrates. This enzyme may also be referred to as cinnamoyl ester hydrolase, ferulic acid esterase or hydroxycinnamoyl esterase. It may also be referred to as a hemicellulase accessory enzyme, since it may help xylanases and pectinases to break down plant cell wall hemicellulose and pectin.

A “pectinase” is any polypeptide which is capable of degrading or modifying pectin. A polypeptide which is capable of degrading pectin is one which is capable of catalyzing the process of breaking down pectin into smaller units, either partially, for example into oligosaccharides, or completely into sugar monomers. A pectinase as described herein may give rise to a mixed population of oligosacchardies and sugar monomers. Such degradation will typically take place by way of a hydrolysis reaction.

An “a-galactosidase” (EC 3.2.1.22) is any polypeptide which is capable of catalyzing the hydrolysis of terminal, non-reducing a-D-galactose residues in a-D-galactosides, including galactose oligosaccharides, galactomannans, galactans and arabinogalactans. Such a polypeptide may also be capable of hydrolyzing a-D-fucosides. This enzyme may also be referred to as melibiase.

A “beta-galactosidase” (EC 3.2.1.23) is any polypeptide which is capable of catalyzing the hydrolysis of terminal non-reducing b-D-galactose residues in b-D-galactosides. Such a polypeptide may also be capable of hydrolyzing a-L-arabinosides. This enzyme may also be referred to as exo-(1->4)-D-galactanase or lactase.

A “beta-mannanase” (EC 3.2.1.78) is any polypeptide which is capable of catalyzing the random hydrolysis of 1,4-D-mannosidic linkages in mannans, galactomannans and glucomannans. This enzyme may also be referred to as mannan endo-1,4-mannosidase or endo-1,4-mannanase.

A “beta-mannosidase” (EC 3.2.1.25) is any polypeptide which is capable of catalyzing the hydrolysis of terminal, non-reducing b-D-mannose residues in b-D-mannosides. This enzyme may also be referred to as mannanase or mannase.

An “endo-polygalacturonase” (EC 3.2.1.15) is any polypeptide which is capable of catalyzing the random hydrolysis of 1,4-a-D-galactosiduronic linkages in pectate and other galacturonans. This enzyme may also be referred to as polygalacturonase pectin depolymerase, pectinase, endopolygalacturonase, pectolase, pectin hydrolase, pectin polygalacturonase, poly-a-1,4-galacturonide glycanohydrolase, endogalacturonase; endo-D-galacturonase or poly(1,4-a-D-galacturonide)glycanohydrolase.

A “pectin methyl esterase” (EC 3.1.1.11) is any enzyme which is capable of catalysing the reaction: pectin+n H2O=n methanol+pectate. The enzyme may also be known as pectinesterase, pectin demethoxylase, pectin methoxylase, pectin methylesterase, pectase, pectinoesterase or pectin pectylhydrolase.

An “endo-galactanase” (EC 3.2.1.89) is any enzyme capable of catalyzing the endohydrolysis of 1,4-D-galactosidic linkages in arabinogalactans. The enzyme may also be known as arabinogalactan endo-1,4-galactosidase, endo-1,4-galactanase, galactanase, arabinogalactanase or arabinogalactan 4-D-galactanohydrolase.

A “pectin acetyl esterase” is defined herein as any enzyme which has an acetyl esterase activity which catalyzes the deacetylation of the acetyl groups at the hydroxyl groups of GalUA residues of pectin.

An “endo-pectin lyase” (EC 4.2.2.10) is any enzyme capable of catalysing the eliminative cleavage of (1 (R)4)-a-D-galacturonan methyl ester to give oligosaccharides with 4-deoxy-6-0-methyl-a-D-galact-4-enuronosyl groups at their non-reducing ends. The enzyme may also be known as pectin lyase, pectin frans-eliminase; endo-pectin lyase, polymethylgalacturonic transeliminase, pectin methyltranseliminase, pectolyase, PL, PNL or PMGL or (1 (R)4)-6-0-methyl-a-D-galacturonan lyase.

A “pectate lyase” (EC 4.2.2.2) is any enzyme capable of catalysing the eliminative cleavage of (1 (R)4)-a-D-galacturonan to give oligosaccharides with 4-deoxy-a-D-galact-4-enuronosyl groups at their non-reducing ends. The enzyme may also be known polygalacturonic transeliminase, pectic acid transeliminase, polygalacturonate lyase, endopectin methyltranseliminase, pectate transeliminase, endogalacturonate transeliminase, pectic acid lyase, pectic lyase, a-1,4-D-endopolygalacturonic acid lyase, PGA lyase, PPase-N, endo-α-1,4-polygalacturonic acid lyase, polygalacturonic acid lyase, pectin frans-eliminase, polygalacturonic acid frans-eliminase or (1 (R)4)-a-D-galacturonan lyase.

An “alpha rhamnosidase” (EC 3.2.1.40) is any polypeptide which is capable of catalysing the hydrolysis of terminal non-reducing a-L-rhamnose residues in a-L-rhamnosides or alternatively in rhamnogalacturonan. This enzyme may also be known as a-L-rhamnosidase T, a-L-rhamnosidase N or a-L-rhamnoside rhamnohydrolase.

An “exo-galacturonase” (EC 3.2.1.82) is any polypeptide capable of hydrolysis of pectic acid from the non-reducing end, releasing digalacturonate. The enzyme may also be known as exo-poly-a-galacturonosidase, exopolygalacturonosidase or exopolygalacturanosidase.

An “exo-galacturonase” (EC 3.2.1.67) is any polypeptide capable of catalysing: (1,4-a-D-galacturonide)n+H2O=(1,4-a-D-galacturonide), -i+D-galacturonate. The enzyme may also be known as galacturan 1,4-a-galacturonidase, exopolygalacturonase, poly(galacturonate) hydrolase, exo-D-galacturonase, exo-D-galacturonanase, exopoly-D-galacturonase or poly(1,4-a-D-galacturonide) galacturonohydrolase.

An “exopolygalacturonate lyase” (EC 4.2.2.9) is any polypeptide capable of catalysing eliminative cleavage of 4-(4-deoxy-a-D-galact-4-enuronosyl)-D-galacturonate from the reducing end of pectate, i.e. de-esterified pectin. This enzyme may be known as pectate disaccharide-lyase, pectate exo-lyase, exopectic acid transeliminase, exopectate lyase, exopolygalacturonic acid-frans-eliminase, PATE, exo-PATE, exo-PGL or (1 (R)4)-a-D-galacturonan reducing-end-disaccharide-lyase.

A “rhamnogalacturonan hydrolase” is any polypeptide which is capable of hydrolyzing the linkage between galactosyluronic acid and rhamnopyranosyl in an endo-fashion in strictly alternating rhamnogalacturonan structures, consisting of the disaccharide [(1,2-alpha-L-rhamnoyl-(1,4)-alpha-galactosyluronic acid].

A “rhamnogalacturonan lyase” is any polypeptide which is any polypeptide which is capable of cleaving a-L-Rhap-(1 (R)4)-a-D-GalpA linkages in an endo-fashion in rhamnogalacturonan by beta-elimination.

A “rhamnogalacturonan acetyl esterase” is any polypeptide which catalyzes the deacetylation of the backbone of alternating rhamnose and galacturonic acid residues in rhamnogalacturonan.

A “rhamnogalacturonan galacturonohydrolase” is any polypeptide which is capable of hydrolyzing galacturonic acid from the non-reducing end of strictly alternating rhamnogalacturonan structures in an exo-fashion.

A “xylogalacturonase” is any polypeptide which acts on xylogalacturonan by cleaving the b-xylose substituted galacturonic acid backbone in an enc/o-manner. This enzyme may also be known as xylogalacturonan hydrolase.

“Ligninase” includes enzymes that can hydrolyze or break down the structure of lignin polymers. Enzymes that can break down lignin include lignin peroxidases, manganese peroxidases, laccases and feruloyl esterases, and other enzymes described in the art known to depolymerize or otherwise break lignin polymers. Also included are enzymes capable of hydrolyzing bonds formed between hemicellulosic sugars (notably arabinose) and lignin. Ligninases include but are not limited to the following group of enzymes: lignin peroxidases (EC 1.11.1.14), manganese peroxidases (EC 1.11.1.13), laccases (EC 1.10.3.2) and feruloyl esterases (EC 3.1.1.73).

“Hexosyltransferase” (2.4.1-) includes enzymes which are capable of catalysing a transferase reaction, but which can also catalyze a hydrolysis reaction, for example of cellulose and/or cellulose degradation products. An example of a hexosyltransferase which may be used is a b-glucanosyltransferase. Such an enzyme may be able to catalyze degradation of (1,3)(1,4) glucan and/or cellulose and/or a cellulose degradation product.

“Phytase” means any type of phosphatase enzyme that catalyzes the hydrolysis of phytic acid (myo-inositol hexakisphosphate) which is an indigestible, organic form of phosphorus that is found in grains and oil seeds, and releases a usable form of inorganic phosphorus.

“Lytic polysaccharide monooxygenases (LPMOs)” are enzymes that have recently been classified by CAZy in family AA9 (Auxiliary Activity Family 9) LPMOs or family AA10 (Auxiliary Activity Family 10) LPMOs. Lytic polysaccharide monooxygenases are able to open a crystalline glucan structure and enhance the action of cellulases on lignocellulose substrates. They are enzymes having cellulolytic enhancing activity. Lytic polysaccharide monooxygenases may also affect cello-oligosaccharides. According to the latest literature, (see Isaksen et al., Journal of Biological Chemistry, vol. 289, no. 5, p. 2632-2642), proteins named GH61 (glycoside hydrolase family 61 or sometimes referred to EGIV) are lytic polysaccharide monooxygenases. GH61 was originally classified as endoglucanase based on measurement of very weak endo-1,4-d-glucanase activity in one family member but have recently been reclassified by CAZy in family AA9. CBM33 (family 33 carbohydrate-binding module) is also a lytic polysaccharide monooxygenase (see Isaksen et al, Journal of Biological Chemistry, vol. 289, no. 5, pp. 2632-2642). CAZy has recently reclassified CBM33 in the AA10 family.

A “lactase” (EC 3.2.1.21) is an enzyme that hydrolyses terminal, nonreducing beta-D-glucosyl residues with release of beta-D-glucose.

A “sucrase” (EC 3.2.1.26 (invertase)) catalyzes the hydrolysis of sucrose into fructose and glucose.

A “lipase” is an enzyme that catalyzes the hydrolysis of fats (lipids). Examples include, but are not limited to triacylglycerol lipases, phospholipases (such as A1, A2, B, C and D), cutinases and galactolipases.

A “protease” is a protein hydrolyzing or modifying proteins. Examples include, but are not limited to, endo-acting proteases (serine proteases, metalloproteases, aspartyl proteases, thiol proteases), exo-acting peptidases that cleave off one amino acid, or dipeptide, tripeptide, etc. from the N-terminal (aminopeptidases) or C-terminal (carboxypeptidases) ends of the polypeptide chain.

“Beta-xylosidases” (EC 3.2.1.37) are polypeptides which are capable of catalysing the hydrolysis of 1,4-p-D-xylans, to remove successive D-xylose residues from the nonreducing termini. Beta-xylosidases may also hydrolyze xylobiose. Beta-xylosidase may also be referred to as xylan 1,4-p-xylosidase, 1,4-p-D-xylan xylohydrolase, exo-1,4-p-xylosidase or xylobiase.

### DESCRIPTION OF THE INVENTION

Milled grain, e.g., preferably corn, is fed to a dry milling step in order to reduce the particle size. In a preferred embodiment, the method in accordance with the invention may be configured in a manner such that the mass fraction of particles with a particle size of <0.5 mm following milling is at least 60 percent, preferably at least 65 percent, particularly preferably at least 70 percent of the milled grain (e.g., corn meal). In a further embodiment, the method in accordance with the invention may be configured in a manner such that the mass fraction of particles with a particle size of <0.36 mm after milling is at least 45 percent, preferably at least 50 percent, particularly preferably at least 55 percent. These significantly smaller particle sizes compared with the prior art have the advantage that the particle surface is more easily accessible to the starch-degrading enzymes. Furthermore, in this manner, settling out of particles in the fermenters can be significantly reduced; this would otherwise lead to residual starch contents in the fermented mash which were too high and to losses of productivity when using a cold mash process.

The milled grain (e.g., corn meal) from the dry milling step is mashed with different fluids. These fluids are at least whole stillage and outflow from the biogas unit. In addition, other fluids may be involved, for example thin stillage or process liquids.

Because the recycle of dry matter in the form of whole stillage into the mashing step is high, the advantages of the energy savings of the cooking stage and mash distillation at low temperatures can be obtained and at the same time, low ethanol yield losses due to residual starch are possible. In a preferred embodiment, the method is configured in a manner such that per tonne of milled grain (e.g., corn meal) in the distillation step, at least 400 liters of ethanol, preferably at least 425 liters of ethanol, particularly preferably at least 435 liters of ethanol are separated. In this regard, at least 0.1 t TS in the form of whole stillage per tonne of milled grain (e.g., corn meal) has to be recycled to the mashing step. In a preferred embodiment, the method in accordance with the invention may be configured in a manner such that at least 0.12 t TS, preferably at least 0.14 t TS, particularly preferably at least 0.16 t TS in the form of whole stillage per tonne of milled grain (e.g., corn meal) is recycled to the mashing step.

In a preferred embodiment, the method in accordance with the invention may be configured in a manner such that a yield enhancing composition comprising at least one, at least two, at least three, at least four, or at least five different enzymes and/or at least one, at least two, at least three, at least four or at least five different microorganisms can also be added to the whole stillage that is recycled to the mashing step. This has the advantage that in this manner, glucose and nitrogen sources from the enzymatic hydrolysis of materials remaining in the whole stillage, e.g., hemicellulose, cellulose, residual starch, protein, yeast cell wall beta-glucan etc.) is made available to the yeasts in the fermentation step, and therefore the ethanol yield can be increased. With the high recycle of whole stillage in accordance with the invention compared with a typical ethanol unit, large quantities of hemicellulose, cellulose, residual starch, protein, yeast cell wall beta-glucan etc. can also be recycled to the mashing step and thus are available for enzymatic hydrolysis and subsequent assimilation by yeast for enhancing fermentation.

In a preferred embodiment, the method in accordance with the invention may be configured in a manner such that biomass is added with the yield enhancing composition to the whole stillage that recycled to the mashing step. The biomass may be pretreated before being added with the yield enhancing composition to the whole stillage that is recycled to the mashing step. Pretreatment methods are known in the art and include, but are not limited to, heat, mechanical, chemical modification, biological modification and any combination thereof. The biomass may also be washed before it is contacted with the yield enhancing composition. The whole stillage and biomass, e.g., pretreated biomass, may be incubated together with the yield enhancing composition before being recycled to the mashing step to initiate the hydrolysis of the hemicellulose, cellulose, residual starch, protein, yeast cell wall beta-glucan, etc. to make it more readily available for the yeast during fermentation.

In a further preferred embodiment, the method in accordance with the invention may be configured in a manner such that at least 0.2 t TS, preferably at least 0.3 t TS, particularly preferably at least 0.4 t TS per tonne of milled grain (e.g., corn meal) is added in the form of a mixture of thin stillage and whole stillage. In a preferred embodiment, the method in accordance with the invention may be configured in a manner such that the mixture of thin stillage and whole stillage contains a volume fraction of at least 10 percent, preferably at least 20 percent, particularly preferably at least 30 percent whole stillage.

In a preferred embodiment, at least 0.1 t TS, preferably at least 0.2 t TS, preferably at least 0.25 t TS thin stillage pert of mash is recycled. Recycling larger quantities of thin stillage is advantageous compared with using fresh water or process liquids, which are obtained in an energy-intensive manner by the evaporation of thin stillage.

In a preferred embodiment, the method is configured in a manner such that per tonne of milled grain (e.g., corn meal), at least 15 kg, preferably at least 18 kg, particularly preferably at least 20 kg of glycerin is recycled to the mashing step via the whole stillage and thin stillage. A high recycle of glycerin significantly reduces the fresh formation of glycerin in the ethanol fermentation step. In this manner, less starch or sugar is lost as glycerin and thus the ethanol yield is increased. In a preferred embodiment, the method is configured in a manner such that the recycle of the mixture of thin stillage and whole stillage is selected to be so high that a mass fraction of less than 2.5 percent, preferably less than 2.3 percent, particularly preferably less than 2.1 percent of the starch and sugar present in the milled grain (e.g., corn meal) is transformed into glycerin. In a preferred embodiment, the method is configured in a manner such that the recycle of the mixture of thin stillage and whole stillage is selected to be so high that less than 19 kg, preferably less than 17 kg, particularly preferably less than 15 kg of glycerin per tonne of milled grain (e.g., corn meal) is produced.

The fraction of outflow from the biogas unit is at least 0.1 m³ per tonne of milled grain (e.g., corn meal). Recycling outflow from the biogas unit has the advantage of economizing on fresh water as well as process liquids. Process liquids are typically obtained hi an energy-intensive manner by the evaporation of thin stillage. The addition of outflow from the biogas unit to the mashing step does not lead to a severe inhibition of the enzymes and yeasts in the ethanol process if the addition of NH4-N in the form of outflow from the biogas unit is limited. In a preferred embodiment, the method is configured in a manner such that in the mashing step, a maximum of 1000 g, preferably a maximum of 800 g, particularly preferably a maximum of 600 g of NH4-N per tonne of milled grain (e.g., corn meal) is recycled via the outflow from the biogas unit. On the other hand, a certain quantity of NH4-IN recycle into the mashing step should be aimed for to reduce or dispense with the use of external nitrogen sources such as urea. In a preferred embodiment, the method is configured in a manner such that in the mashing step, at least 100 g, preferably at least 200 g, particularly preferably at least 400 g of ammonium nitrogen per tonne of milled grain (e.g., corn meal) is recycled via the outflow from the biogas unit. In a preferred embodiment, the fraction of outflow from the biogas unit per tonne of milled grain (e.g., corn meal) is at least 0.2 m³, preferably at least 0.4 m³, particularly preferably at least 0.8 m³.

In a preferred embodiment, the method in accordance with the invention may be configured in a manner such that a yield enhancing composition comprising at least one, at least two, at least three, at least four, or at least five different enzymes and/or at least one, at least two, at least three, at least four or at least five different microorganisms can also be fed from the biogas unit to the mashing step via the outflow from the biogas unit. This has the advantage that in this manner, glucose from the enzymatic hydrolysis of materials remaining after anaerobic digestion of the whole stillage and/or biomass, e.g., hemicellulose, cellulose, residual starch, protein, yeast cell wall beta-glucan, etc.) is made available to the yeasts in the fermentation step, and therefore the ethanol yield can be increased. With the high recycle of whole stillage in accordance with the invention compared with a typical ethanol unit, large quantities of hemicellulose, cellulose, residual starch, protein, yeast cell wall beta-glucan, etc. can also be recycled to the mashing step and thus are available for enzymatic hydrolysis.

Preferably, cellulose is present in a mass fraction of at least 5 percent of the TS of the whole stillage. In an embodiment, the cellulose is present in a mass fraction of from 5 percent to 20 percent of the TS of the whole stillage. The recycle of at least 0.1 t TS to the mashing step therefore recycles at least 5 kg of cellulose. In an embodiment, the recycle of at least 0.1 t TS to the mashing step recycles from 5 kg to 20 kg of cellulose. The cellulose content in the whole stillage is determined by means of an animal feed analysis in accordance with VDLUFA III. The cellulose content is calculated as the measured ADF value (“acid detergent fiber”) minus the measured ADL value (“acid detergent lignin”) of a whole stillage sample.

Preferably, hemicellulose is present in a mass fraction of at least 5 percent of the TS of the whole stillage. In an embodiment, the hemicellulose is present in a mass fraction of from 5 percent to 20 percent of the TS of the whole stillage. The recycle of at least 0.1 t TS to the mashing step therefore recycles at least 5 kg of hemicellulose. In an embodiment, the recycle of at least 0.1 t TS to the mashing step recycles from 5 kg to 20 kg of hemicellulose. Methods of determining the hemicellulose content in the whole stillage are well known to those skilled in the art.

Preferably, residual starch is present in a mass fraction of at least 2 percent of the TS of the whole stillage. In an embodiment, the residual starch is present in a mass fraction of from 2 percent to 10% of the TS of the whole stillage. The recycle of at least 0.1 t TS to the mashing step therefore recycles at least 2 kg of residual starch. In an embodiment, the recycle of at least 0.1 T TS to the mashing step recycles from 2 kg to 10 kg of residual starch. Methods of calculating total residual starch are well known to those skilled in the art. A commercial kit for determining residual starch is available for purchase from MEGAZYME®.

Preferably, protein is present in a mass fraction of at least 6 percent of the TS of the whole stillage. In an embodiment, the protein is present in a mass fraction of from 6 percent to 30 percent of the TS of the whole stillage. The recycle of at least 0.1 t TS to the mashing step therefore recycles at least 6 kg of protein. In an embodiment, the recycle of at least 0.1 t TS to the mashing step recycles from 6 kg to 30 kg of protein. When protease are added to the whole stillage prior to recycle, the proteases can break down the protein into amino acids and short oligopeptides that provide a source of free amino nitrogen that is readily assimilable by yeast to fuel yeast growth and enhance fermentation performance. Methods of determining protein content in the whole stillage are well known to those skilled in the art. The residual protein content may be calculated as (protein=nitrogenx6.25) using a LECO FP628 nitrogen analyzer. Preferably, beta-glucan from yeast cell walls is present in a mass fraction of at least 1 percent of the TS of the whole stillage. In an embodiment, beta-glucan from yeast cell walls is present in a mass fraction of from 1 percent to 10 percent. The recycle of at least 0.1 t TS to the mashing step therefore recycles at least 1 kg of yeast cell wall beta-glucan. The beta-glucanases disclosed herein can be used to hydrolyze the yeast cell wall beta-glucan present in the whole stillage to glucose. Methods for determining yeast cell wall beta-glucan content in the whole stillage are well known to those skilled in the art.

In a further embodiment, the method in accordance with the invention may be configured in a manner such that process liquids in addition to whole stillage and outflow from the biogas unit are used in the mashing step. The process liquids in this regard may be selected from the group comprising: thin stillage, singlings, untreated water, drinking water, water for industrial use, rainwater, ground water, surface water, condensates from the evaporation of thin stillage, process water from CO₂ scrubbers, blowdown water from cooling towers, blowdown water and blow-off water from steam production boilers, and mixtures thereof. In a preferred embodiment, the method in accordance with the invention may be configured in a manner such that less than 1.2 m³, preferably less than 0.8 m³, particularly preferably less than 0.2 m³ of fresh water is used per tonne of milled grain (e.g., corn meal). In such embodiment, a yield enhancing composition comprising at least one, at least two, at least three, at least four, or at least five different enzymes and/or at least one, at least two, at least three, at least four or at least five different microorganisms may be added to the process liquids, the whole stillage, and/or the outflow from the biogas unit.

In a preferred embodiment, the method in accordance with the invention may be configured in a manner such that the pH of the mash is adjusted to less than 4.5, preferably to less than 4.2, particularly preferably to less than 3.8. Despite the cold mash process, contamination by bacteria in the ethanol fermentation can be avoided, proteins are concentrated better, and the availability of cellulose in the milled grain (e.g., corn meal) for biological degradation in the ethanol unit and biogas unit can be enhanced.

The mash undergoes a cold mash process, i.e. a cooking stage is carried out in which the mash is heated to temperatures below the gelatinization temperature of the starch in the milled grain (e.g., corn meal). This results in significant energy savings compared with typical cooking stages at 104-107 degrees centigrade. In a preferred embodiment, the method in accordance with the invention may be configured in a manner such that the mash is heated to a maximum of 70 degrees centigrade, preferably to a maximum of 66 degrees centigrade, particularly preferably to a maximum of 64 degrees centigrade.

The mash from the cooking stage undergoes a fermentation step in which ethanol is formed. The ethanol-containing mash from the fermentation is then fed to a distillation step in which ethanol is separated out. This results in significant energy savings compared with typical mash distillation steps at approximately 108 degrees centigrade. In a preferred embodiment, the method in accordance with the invention may be configured in a manner such that the ethanol-containing mash is heated to a maximum of 87 degrees centigrade, preferably to a maximum of 79 degrees centigrade, particularly preferably to a maximum of 68 degrees centigrade In addition to saving energy, the low temperatures of the cooking stage and distillation step also mean that higher quality proteins are present in the whole stillage. This can be used to obtain high value animal feed and foodstuffs from the whole stillage or thin stillage.

The ethanol-depleted mash from the distillation step, what is known as the whole stillage, is re-used in several different ways. A portion is recycled directly to the mashing step. A further portion is fed directly to the biogas unit. The direct use of whole stillage has the following advantages over the typical solid-liquid separation of the entirety of the whole stillage: on the one hand, electrical energy for the solid-liquid separation is saved. On the other hand, a large proportion of the residual starch from the fermentation is recycled; as animal feed or as a substrate for the biogas unit, it would no longer be available for ethanol formation.

In a preferred embodiment, the method in accordance with the invention may be configured in a manner such that a further portion of the whole stillage is fed to a solid-liquid separation step in order to produce thin stillage. In a preferred embodiment, the method in accordance with the invention may be configured in a manner such that a maximum volume fraction of 70 percent, preferably a maximum of 50 percent, particularly preferably a maximum of 30 percent of the whole stillage is fed to a solid-liquid separation step to generate thin stillage and wet cake. As an example, the thin stillage may be used in the mashing step, but also for obtaining corn oil, animal feed or foodstuffs. In a further preferred embodiment, the method in accordance with the invention may be configured in a manner such that a further portion of the whole stillage is used as animal feed. The wet cake may also be used as animal feed. In a further preferred embodiment, a maximum of 0.18 t TS, preferably a maximum of 0.12 t TS, particularly preferably a maximum of 0.06 t TS of animal feed per tonne of milled grain (e.g., corn meal) selected from the group comprising whole stillage, wet cake, syrup (thin stillage concentrate) and their dried forms are produced. In a preferred embodiment, the method in accordance with the invention may be configured in a manner such that the wet cake is fed to the biogas unit.

In a further preferred embodiment, the method in accordance with the invention may be configured in a manner such that a protein product with a raw protein content (mass fraction of raw protein with respect to TS protein product) of more than 44 percent is obtained from the thin stillage. In a further preferred embodiment, the method in accordance with the invention may be configured in a manner such that a protein product with a raw protein content (mass fraction of raw protein with respect to TS protein product) of more than 70 percent is obtained from thin stillage. In a further embodiment, the method in accordance with the invention may be configured in a manner such that corn oil is obtained from thin stillage. In a preferred embodiment, the residual materials from the production of protein products and residual materials from the production of corn oil are fed to the biogas unit.

The biogas unit consists of at least one biogas fermenter of the continuous stirred tank reactor (CTSR) type, in which components of the substrate which is fed in are transformed by a mixed culture of bacteria and archaea to form biogas and by-products such as ammonium. The substrate which is fed in contains at least whole stillage, and in a preferred embodiment may also contain wet cake as well as residual substances from the production of corn oil and from the production of protein products.

In a preferred embodiment, the method in accordance with the invention may be configured in a manner such that a yield enhancing composition comprising at least one, at least two, at least three, at least four, or at least five different enzymes and/or at least one, at least two, at least three, at least four or at least five different microorganisms can also be added to the whole stillage that is fed to the biogas unit.

In a preferred embodiment, the method in accordance with the invention may be configured in a manner such that a yield enhancing composition comprising at least one, at least two, at least three, at least four, or at least five different enzymes and/or at least one, at least two, at least three, at least four or at least five different microorganisms can also be added to the wet cake that is fed to the biogas unit.

In a preferred embodiment, the method in accordance with the invention may be configured in a manner such that a yield enhancing composition comprising at least one, at least two, at least three, at least four, or at least five different enzymes and/or at least one, at least two, at least three, at least four or at least five different microorganisms can also be added to the residual materials from the production of protein products that is fed to the biogas unit.

In a preferred embodiment, the method in accordance with the invention may be configured in a manner such that a yield enhancing composition comprising at least one, at least two, at least three, at least four, or at least five different enzymes and/or at least one, at least two, at least three, at least four or at least five different microorganisms can also be added to the residual materials from the production of corn oil that is fed to the biogas unit.

In a preferred embodiment, the method in accordance with the invention may be configured in a manner such that a yield enhancing composition comprising at least one, at least two, at least three, at least four, or at least five different enzymes and/or at least one, at least two, at least three, at least four or at least five different microorganisms is added to the biogas unit.

Depending on the selected process parameters, specific unwanted metabolites may accumulate during the anaerobic fermentation. These are, inter alia, organic acids such as, for example, acetic acid, propionic acid, butyric acid, isobutyric acid, valeric acid, isovaleric acid and caproic acids, and aromatic components such as, for example, phenol, indole, skatole or cresols. They could have a negative effect on the ethanol fermentation in the ethanol unit and result in odor-related problems.

High ammonium concentrations can inhibit anaerobic fermentation. Typically, then, the NH4-N content is regulated to less than 6000 μm, for example by dilution with process liquids. However, dilution drops the mean residence time of the whole stillage to be fermented in the biogas fermenter, which could result in an unwanted drop in the biogas yield or an accumulation of metabolites. A stable and efficient anaerobic fermentation of whole stillage is also possible at higher ammonium concentrations. This has the advantage that over-dilution and the associated reduction in the residence time, energy consumption and water consumption are reduced. In a preferred embodiment, the method in accordance with the invention may be configured in a manner such that the ammonium concentration in the biogas fermenters is adjusted to between 6000-9000 ppm, preferably to between 7000-9000 ppm, particularly preferably to between 7500-9000 ppm. In a further preferred embodiment, the method in accordance with the invention may be configured in a manner such that the temperature in the biogas fermenters in the biogas unit is adjusted to below 43 degrees centigrade. This is advantageous, because at higher temperatures, the chemical equilibrium is displaced from ammonium to ammonia and ammonia has a more inhibiting action on anaerobic fermentation.

Even at high ammonium concentrations, a stable fermentation as well as low contents of unwanted metabolites can be obtained by a cascade configuration of biogas fermenters. In a preferred embodiment, the method in accordance with the invention may be configured in a manner such that at least three, preferably at least four biogas fermenters are operated in a cascade in the biogas unit. Infeeding is carried out into the first two stages of the cascade or preferably, into only the first stage of the cascade.

To guarantee a stable fermentation as well as low unwanted metabolite contents even at high ammonium concentrations, a sufficiently long mean hydraulic residence time in the biogas unit is vital. In a preferred embodiment, the method in accordance with the invention may be configured in a manner such that the mean hydraulic residence time for the biogas unit is at least 30 days, preferably at least 50 days, particularly preferably at least 70 days. In a preferred embodiment, the method in accordance with the invention may be configured in a manner such that the hydraulic residence time is selected to be as long as is required for the concentration of organic acids and aromatic compounds in the outflow from the biogas unit to be a maximum of 450 ppm respectively, preferably a maximum of 300 ppm respectively, particularly preferably a maximum of 150 ppm respectively.

In a further embodiment, the method in accordance with the invention may be configured in a manner such that the outflow from the biogas fermenter undergoes solid-liquid separation and the solid phase, the outflow solids, is discharged. In a preferred embodiment, the outflow solids are used for fertilizer and soil improvement.

In a preferred embodiment, the method in accordance with the invention may be configured in a manner such that the outflow from the biogas fermenter or, preferably, the liquid phase from solid-liquid separation of the outflow from the biogas fermenter, is fed to an ammonia stripping step and the NH4-N content of the liquid phase is reduced to less than 1000 ppm, preferably to less than 750 ppm, particularly preferably to less than 500 ppm. This has the advantage that more outflow from the biogas unit can be recycled to the mashing step without compromising the ethanol fermentation.

In a further embodiment, the method in accordance with the invention may be configured in a manner such that the liquid, ammonium-depleted phase from the ammonia stripping step undergoes an evaporation step. In a preferred embodiment, 0.7 t, preferably 0.8 t, particularly preferably 0.9 t of water per tonne of liquid phase is removed from the liquid, ammonium-depleted phase as an evaporation condensate. This high water extraction is advantageous in that a nutrient-rich concentrate, hereinafter termed the nutrient concentrate, is produced which can be used as a valuable fertilizer. Furthermore, less suspended dry matter is recycled to the ethanol unit via the outflow from the biogas unit, which reduces the viscosity of the mash or gives rise to larger quantities of milled grain (e.g., corn meal) or a higher recycle of whole stillage and/or thin stillage. Evaporation of this type is also energetically advantageous compared with typical thin stillage evaporation for the production of process water, because more efficient heat recovery can be carried out.

In a further preferred embodiment, the method in accordance with the invention may be configured in a manner such that biomass can be fed to the biogas unit as a substrate in addition to stillage, the wet cake or the residual substances from the ethanol unit. Compared with mono-fermentation of these substances, this has the advantage that the addition of nutrients such as, for example, sodium or nitrogen which are vital to the growth of microorganisms in the biogas unit, is reduced, because these are already present in the residual substances from the ethanol unit.

The biomass can be pretreated before it is contacted with the yield enhancing composition. Pretreatment methods are known in the art and include, but are not limited to, heat, mechanical, chemical modification, biological modification and any combination thereof. The biomass may also be washed before it is contacted with the yield enhancing composition.

In a further embodiment, the method in accordance with the invention may be configured in a manner such that a portion of the biogas from the biogas unit is used for the production of process energy for the ethanol unit and/or the biogas unit. As an example, biogas can be converted into electrical process energy in a co-generation unit. As a further example, biogas can be used in a steam boiler for the production of steam. In a preferred embodiment, the method in accordance with the invention may be configured in a manner such that the entire steam requirement for the combined ethanol unit and biogas unit can be provided by biogas produced in the biogas unit. In a preferred embodiment, the method in accordance with the invention may be configured in a manner such that the entire energy requirement for the combined ethanol unit and biogas unit is provided by the biogas produced in the biogas unit, wherein the usual units of the prior art may be used for the production of electrical energy and steam from biogas.

In a preferred embodiment, the combination of a bioethanol unit and a biogas unit may be configured in a manner such that a maximum of 0.5 m³, preferably a maximum of 0.2 m³, particularly preferably a maximum of 0.1 m³ of effluent is produced per tonne of milled grain (e.g., corn meal), which effluent is discharged from the combined bioethanol unit and biogas unit.

In a preferred embodiment, the method in accordance with the invention is configured in a manner such that the biogas is fed to a biogas purification step in which at least CO₂ and biomethane are obtained as products. As an example, the biomethane may be compressed and fed into a natural gas grid or used as a vehicle fuel.

### Yield Enhancing Composition of the Present Invention

As noted above, a yield enhancing composition comprising at least one, at least two, at least three, at least four, or at least five different types of enzymes and/or at least one, at least two, at least three, or at least four different microbial strains may be:
i) added to the whole stillage that is fed to the mashing step; and/or
ii) added to the whole stillage that is fed to the biogas unit; and/or
iii) added to the outflow of the biogas unit before being mashed with the corn meal in the mashing step; and/or
iv) added to the thin stillage that is fed to the mashing step; and/or
v) added to the residual materials resulting from purification of the corn oil that are fed to the biogas unit; and/or
vi) added to the residual materials resulting from purification of the protein product that are fed to the biogas unit; and/or
vii) added to the wet cake that is fed to the mashing step; and/or
viii) added to the wet cake that is fed to the biogas unit; and/or
ix) added to the biogas unit; and/or
x) biomass added to any one of i)-ix).

The yield enhancing composition may be formulated to enhance biogas yield, ethanol yield, or ethanol yield and biogas yield. The skilled artisan will appreciate that the manner in which the yield enhancing composition is configured to enhance biogas yield, ethanol yield, or ethanol yield and biogas yield depends on a variety of factors, including for example, where the yield enhancing composition is being added (e.g., as outlined in i), ii), iii), iv), v), vi), vii), viii), ix), etc.), the composition of the material to which it is added (e.g., cellulose, hemicellulose, protein, fat, yeast cell wall beta-glucan, residual starch, etc.), the type of milled grain, whether additional biomass is added, the nature and extent to which the biomass has been pre-treated, etc.

The yield enhancing composition may include at least one, at least two, at least three, at least four, or at least five different types of enzymes. The enzymes may be fungal enzymes, bacterial enzymes, or archaeal enzymes. Examples of different types of enzymes suitable for use in the yield enhancing composition include, but are not limited to, an amylase, cellulases, such as a cellobiohydrolase, an endo-p-1,4-glucanase, and/or a beta-glucosidase, beta-glucanases, such as a beta-(1,3)-glucanase, a beta-(1,4)-glucanase a beta-(1,3)(1,4)-glucanase, and/or a beta-(1,6)-glucanase, hemicellulases, for example, an endoxylanase, a beta-xylosidase, a arabinofuranosidase (e.g., alpha-L-arabionofuranosidase), a glucuronidase (e.g., an alpha-D-glucuronidase), an acetyl xylan esterase, a feruloyl esterase, a coumaroyl esterase, an alpha-galactosidase, a beta-galactosidase, a beta-mannanase and/or a beta-mannosidase, a lytic polysaccharide monooxygenase (e.g. GH61), pectinases, for example, an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and/or a xylogalacturonase, a protease, a lipase, a phytase, a ligninase, a hexosyltransferase, a glucuronidase, an expansin, a cellulose induced protein or a cellulose integrating protein or like protein, a lactase, a sucrase, and a catalase. In an embodiment, the yield enhancing composition comprises at least one enzyme selected from the group consisting an amylase, cellulases, such as a cellobiohydrolase, an endo-p-1,4-glucanase, and/or a beta-glucosidase, beta-glucanases, such as a beta-(1,3)-glucanase, a beta-(1,4)-glucanase a beta-(1,3)(1,4)-glucanase, and/or a beta-(1,6)-glucanase, hemicellulases, for example, an endoxylanase, a beta-xylosidase, a arabinofuranosidase (e.g., alpha-L-arabionofuranosidase), a glucuronidase (e.g., an alpha-D-glucuronidase), an acetyl xylan esterase, a feruloyl esterase, a coumaroyl esterase, an alpha-galactosidase, a beta-galactosidase, a beta-mannanase and/or a beta-mannosidase, a lytic polysaccharide monooxygenase (e.g. GH61), pectinases, for example, an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and/or a xylogalacturonase, a protease, a lipase, a phytase, a ligninase, a hexosyltransferase, a glucuronidase, an expansin, a cellulose induced protein or a cellulose integrating protein or like protein, a lactase, a sucrase, a catalase. In an embodiment, the yield enhancing composition comprises at least two different types of enzymes selected from the group consisting an amylase, cellulases, such as a cellobiohydrolase, an endo-p-1,4-glucanase, and/or a beta-glucosidase, beta-glucanases, such as a beta-(1,3)-glucanase, a beta-(1,4)-glucanase a beta-(1,3)(1,4)-glucanase, and/or a beta-(1,6)-glucanase, hemicellulases, for example, an endoxylanase, a beta-xylosidase, a arabinofuranosidase (e.g., alpha-L-arabionofuranosidase), a glucuronidase (e.g., an alpha-D-glucuronidase), an acetyl xylan esterase, a feruloyl esterase, a coumaroyl esterase, an alpha-galactosidase, a beta-galactosidase, a beta-mannanase and/or a beta-mannosidase, a lytic polysaccharide monooxygenase (e.g. GH61), pectinases, for example, an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and/or a xylogalacturonase, a protease, a lipase, a phytase, a ligninase, a hexosyltransferase, a glucuronidase, an expansin, a cellulose induced protein or a cellulose integrating protein or like protein, a lactase, a sucrase, a catalase. In an embodiment, the yield enhancing composition comprises at least three different types of enzymes selected from the group consisting an amylase, cellulases, such as a cellobiohydrolase, an endo-p-1,4-glucanase, and/or a beta-glucosidase, beta-glucanases, such as a beta-(1,3)-glucanase, a beta-(1,4)-glucanase a beta-(1,3)(1,4)-glucanase, and/or a beta-(1,6)-glucanase, hemicellulases, for example, an endoxylanase, a beta-xylosidase, a arabinofuranosidase (e.g., alpha-L-arabionofuranosidase), a glucuronidase (e.g., an alpha-D-glucuronidase), an acetyl xylan esterase, a feruloyl esterase, a coumaroyl esterase, an alpha-galactosidase, a beta-galactosidase, a beta-mannanase and/or a beta-mannosidase, a lytic polysaccharide monooxygenase (e.g. GH61), pectinases, for example, an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and/or a xylogalacturonase, a protease, a lipase, a phytase, a ligninase, a hexosyltransferase, a glucuronidase, an expansin, a cellulose induced protein or a cellulose integrating protein or like protein, a lactase, a sucrase, a catalase. In an embodiment, the yield enhancing composition comprises at least four different types of enzymes selected from the group consisting an amylase, cellulases, such as a cellobiohydrolase, an endo-p-1,4-glucanase, and/or a beta-glucosidase, beta-glucanases, such as a beta-(1,3)-glucanase, a beta-(1,4)-glucanase a beta-(1,3)(1,4)-glucanase, and/or a beta-(1,6)-glucanase, hemicellulases, for example, an endoxylanase, a beta-xylosidase, a arabinofuranosidase (e.g., alpha-L-arabionofuranosidase), a glucuronidase (e.g., an alpha-D-glucuronidase), an acetyl xylan esterase, a feruloyl esterase, a coumaroyl esterase, an alpha-galactosidase, a beta-galactosidase, a beta-mannanase and/or a beta-mannosidase, a lytic polysaccharide monooxygenase (e.g. GH61), pectinases, for example, an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and/or a xylogalacturonase, a protease, a lipase, a phytase, a ligninase, a hexosyltransferase, a glucuronidase, an expansin, a cellulose induced protein or a cellulose integrating protein or like protein, a lactase, a sucrase, a catalase. In an embodiment, the yield enhancing composition comprises at least five different types of enzymes selected from the group consisting an amylase, cellulases, such as a cellobiohydrolase, an endo-p-1,4-glucanase, and/or a beta-glucosidase, beta-glucanases, such as a beta-(1,3)-glucanase, a beta-(1,4)-glucanase a beta-(1,3)(1,4)-glucanase, and/or a beta-(1,6)-glucanase, hemicellulases, for example, an endoxylanase, a beta-xylosidase, a arabinofuranosidase (e.g., alpha-L-arabionofuranosidase), a glucuronidase (e.g., an alpha-D-glucuronidase), an acetyl xylan esterase, a feruloyl esterase, a coumaroyl esterase, an alpha-galactosidase, a beta-galactosidase, a beta-mannanase and/or a beta-mannosidase, a lytic polysaccharide monooxygenase (e.g. GH61), pectinases, for example, an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and/or a xylogalacturonase, a protease, a lipase, a phytase, a ligninase, a hexosyltransferase, a glucuronidase, an expansin, a cellulose induced protein or a cellulose integrating protein or like protein, a lactase, a sucrase, a catalase. In an embodiment, the yield enhancing composition comprises at least six different types of enzymes selected from the group consisting an amylase, cellulases, such as a cellobiohydrolase, an endo-p-1,4-glucanase, and/or a beta-glucosidase, beta-glucanases, such as a beta-(1,3)-glucanase, a beta-(1,4)-glucanase a beta-(1,3)(1,4)-glucanase, and/or a beta-(1,6)-glucanase, hemicellulases, for example, an endoxylanase, a beta-xylosidase, a arabinofuranosidase (e.g., alpha-L-arabionofuranosidase), a glucuronidase (e.g., an alpha-D-glucuronidase), an acetyl xylan esterase, a feruloyl esterase, a coumaroyl esterase, an alpha-galactosidase, a beta-galactosidase, a beta-mannanase and/or a beta-mannosidase, a lytic polysaccharide monooxygenase (e.g. GH61), pectinases, for example, an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and/or a xylogalacturonase, a protease, a lipase, a phytase, a ligninase, a hexosyltransferase, a glucuronidase, an expansin, a cellulose induced protein or a cellulose integrating protein or like protein, a lactase, a sucrase, a catalase. In an embodiment, the yield enhancing composition comprises at least seven different types of enzymes selected from the group consisting an amylase, cellulases, such as a cellobiohydrolase, an endo-p-1,4-glucanase, and/or a beta-glucosidase, beta-glucanases, such as a beta-(1,3)-glucanase, a beta-(1,4)-glucanase a beta-(1,3)(1,4)-glucanase, and/or a beta-(1,6)-glucanase, hemicellulases, for example, an endoxylanase, a beta-xylosidase, a arabinofuranosidase (e.g., alpha-L-arabionofuranosidase), a glucuronidase (e.g., an alpha-D-glucuronidase), an acetyl xylan esterase, a feruloyl esterase, a coumaroyl esterase, an alpha-galactosidase, a beta-galactosidase, a beta-mannanase and/or a beta-mannosidase, a lytic polysaccharide monooxygenase (e.g. GH61), pectinases, for example, an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and/or a xylogalacturonase, a protease, a lipase, a phytase, a ligninase, a hexosyltransferase, a glucuronidase, an expansin, a cellulose induced protein or a cellulose integrating protein or like protein, a lactase, a sucrase, a catalase. In an embodiment, the yield enhancing composition comprises at least eight different types of enzymes selected from the group consisting an amylase, cellulases, such as a cellobiohydrolase, an endo-p-1,4-glucanase, and/or a beta-glucosidase, beta-glucanases, such as a beta-(1,3)-glucanase, a beta-(1,4)-glucanase a beta-(1,3)(1,4)-glucanase, and/or a beta-(1,6)-glucanase, hemicellulases, for example, an endoxylanase, a beta-xylosidase, a arabinofuranosidase (e.g., alpha-L-arabionofuranosidase), a glucuronidase (e.g., an alpha-D-glucuronidase), an acetyl xylan esterase, a feruloyl esterase, a coumaroyl esterase, an alpha-galactosidase, a beta-galactosidase, a beta-mannanase and/or a beta-mannosidase, a lytic polysaccharide monooxygenase (e.g. GH61), pectinases, for example, an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and/or a xylogalacturonase, a protease, a lipase, a phytase, a ligninase, a hexosyltransferase, a glucuronidase, an expansin, a cellulose induced protein or a cellulose integrating protein or like protein, a lactase, a sucrase, a catalase. In an embodiment, the yield enhancing composition comprises at least nine different types of enzymes selected from the group consisting an amylase, cellulases, such as a cellobiohydrolase, an endo-p-1,4-glucanase, and/or a beta-glucosidase, beta-glucanases, such as a beta-(1,3)-glucanase, a beta-(1,4)-glucanase a beta-(1,3)(1,4)-glucanase, and/or a beta-(1,6)-glucanase, hemicellulases, for example, an endoxylanase, a beta-xylosidase, a arabinofuranosidase (e.g., alpha-L-arabionofuranosidase), a glucuronidase (e.g., an alpha-D-glucuronidase), an acetyl xylan esterase, a feruloyl esterase, a coumaroyl esterase, an alpha-galactosidase, a beta-galactosidase, a beta-mannanase and/or a beta-mannosidase, a lytic polysaccharide monooxygenase (e.g. GH61), pectinases, for example, an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and/or a xylogalacturonase, a protease, a lipase, a phytase, a ligninase, a hexosyltransferase, a glucuronidase, an expansin, a cellulose induced protein or a cellulose integrating protein or like protein, a lactase, a sucrase, a catalase. In an embodiment, the yield enhancing composition comprises at least ten different types of enzymes selected from the group consisting an amylase, cellulases, such as a cellobiohydrolase, an endo-p-1,4-glucanase, and/or a beta-glucosidase, beta-glucanases, such as a beta-(1,3)-glucanase, a beta-(1,4)-glucanase a beta-(1,3)(1,4)-glucanase, and/or a beta-(1,6)-glucanase, hemicellulases, for example, an endoxylanase, a beta-xylosidase, a arabinofuranosidase (e.g., alpha-L-arabionofuranosidase), a glucuronidase (e.g., an alpha-D-glucuronidase), an acetyl xylan esterase, a feruloyl esterase, a coumaroyl esterase, an alpha-galactosidase, a beta-galactosidase, a beta-mannanase and/or a beta-mannosidase, a lytic polysaccharide monooxygenase (e.g. GH61), pectinases, for example, an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and/or a xylogalacturonase, a protease, a lipase, a phytase, a ligninase, a hexosyltransferase, a glucuronidase, an expansin, a cellulose induced protein or a cellulose integrating protein or like protein, a lactase, a sucrase, a catalase. The preceding yield enhancing compositions comprising cellulases and hemicellulases may further include at least one, at least two, at least three, at least four, or at least five different microorganisms (e.g., microbial strains, bacterial strains, mixtures, etc.).

In a preferred embodiment, the yield enhancing composition comprises a cellulase and a hemicellulase. In a preferred embodiment, the yield enhancing composition comprises a cellulase and a hemicellulase, wherein the cellulase is selected from the group consisting of a cellobiohydrolase, an endoglucanase, and a beta-glucosidase and wherein the hemicellulase is selected from the group consisting of endoxylanase, a beta-xylosidase, a arabinofuranosidase a glucuronidase, an acetyl xylan esterase, a feruloyl esterase, a coumaroyl esterase, an alpha-galactosidase, a beta-galactosidase, a beta-mannanase and/or a beta-mannosidase.

In an embodiment, the yield enhancing composition comprises at least two cellulases and at least one hemicellulase, wherein the cellulases are selected from the group consisting of a cellobiohydrolase, an endoglucanase, and a beta-glucosidase and wherein the hemicellulase is selected from the group consisting of endoxylanase, a beta-xylosidase, a arabinofuranosidase a glucuronidase, an acetyl xylan esterase, a feruloyl esterase, a coumaroyl esterase, an alpha-galactosidase, a beta-galactosidase, a beta-mannanase and/or a beta-mannosidase.

In an embodiment, the yield enhancing composition comprises at least three cellulases and at least one hemicellulase, wherein the cellulases comprise a cellobiohydrolase, an endoglucanase, and a beta-glucosidase and wherein the hemicellulase is selected from the group consisting of endoxylanase, a beta-xylosidase, a arabinofuranosidase a glucuronidase, an acetyl xylan esterase, a feruloyl esterase, a coumaroyl esterase, an alpha-galactosidase, a beta-galactosidase, a beta-mannanase and/or a beta-mannosidase.

In an embodiment, the yield enhancing composition comprises at least two cellulases and at least two hemicellulases, wherein the cellulases are selected from the group consisting of a cellobiohydrolase, an endoglucanase, and a beta-glucosidase and wherein the hemicellulases are selected from the group consisting of endoxylanase, a beta-xylosidase, a arabinofuranosidase a glucuronidase, an acetyl xylan esterase, a feruloyl esterase, a coumaroyl esterase, an alpha-galactosidase, a beta-galactosidase, a beta-mannanase and/or a beta-mannosidase.

In an embodiment, the yield enhancing composition comprises at least three cellulases and at least two hemicellulases, wherein the cellulases are selected from the group consisting of a cellobiohydrolase, an endoglucanase, and a beta-glucosidase and wherein the hemicellulases are selected from the group consisting of endoxylanase, a beta-xylosidase, a arabinofuranosidase a glucuronidase, an acetyl xylan esterase, a feruloyl esterase, a coumaroyl esterase, an alpha-galactosidase, a beta-galactosidase, a beta-mannanase and/or a beta-mannosidase.

In an embodiment, the yield enhancing composition comprises at least two cellulases and at least three hemicellulases, wherein the cellulases are selected from the group consisting of a cellobiohydrolase, an endoglucanase, and a beta-glucosidase and wherein the hemicellulases are selected from the group consisting of endoxylanase, a beta-xylosidase, a arabinofuranosidase a glucuronidase, an acetyl xylan esterase, a feruloyl esterase, a coumaroyl esterase, an alpha-galactosidase, a beta-galactosidase, a beta-mannanase and/or a beta-mannosidase.

In an embodiment, the yield enhancing composition comprises at least three cellulases and at least three hemicellulases, wherein the cellulases are selected from the group consisting of a cellobiohydrolase, an endoglucanase, and a beta-glucosidase and wherein the hemicellulases are selected from the group consisting of endoxylanase, a beta-xylosidase, a arabinofuranosidase a glucuronidase, an acetyl xylan esterase, a feruloyl esterase, a coumaroyl esterase, an alpha-galactosidase, a beta-galactosidase, a beta-mannanase and/or a beta-mannosidase.

In an embodiment, the yield enhancing composition comprises at least four cellulases and at least one hemicellulases, wherein the cellulases are selected from the group consisting of a cellobiohydrolase I, a cellobiohydrolase II, an endoglucanase I, an endoglucanase II, and a beta-glucosidase and wherein the hemicellulases are selected from the group consisting of endoxylanase, a beta-xylosidase, a aarabinofuranosidase a glucuronidase, an acetyl xylan esterase, a feruloyl esterase, a coumaroyl esterase, an alpha-galactosidase, a beta-galactosidase, a beta-mannanase and/or a beta-mannosidase.

In an embodiment, the yield enhancing composition comprises at least five cellulases and at least one hemicellulase, wherein the cellulases comprise a cellobiohydrolase I, a cellobiohydrolase II, an endoglucanase I, an endoglucanase II, and a beta-glucosidase and wherein the hemicellulases are selected from the group consisting of endoxylanase, a beta-xylosidase, a arabinofuranosidase a glucuronidase, an acetyl xylan esterase, a feruloyl esterase, a coumaroyl esterase, an alpha-galactosidase, a beta-galactosidase, a beta-mannanase and/or a beta-mannosidase.

In a preferred embodiment, the yield enhancing composition comprises a cellulase and a xylanase. In an embodiment, the yield enhancing composition comprises a cellobiohydrolase and a xylanase. In an embodiment, the yield enhancing composition comprises a beta-glucosidase and a xylanase. In an embodiment, the yield enhancing composition comprises an endoglucanase and a xylanase. In an embodiment, the yield enhancing composition comprises a cellobiohydrolase, a beta-glucosidase and a xylanase. In an embodiment, the yield enhancing composition comprises a cellobiohydrolase, an endoglucanse and a xylanase. In an embodiment, the yield enhancing composition comprises an endoglucanase, a beta-glucosidase and a xylanase. In an embodiment, the yield enhancing composition comprises a cellobiohydrolase, a beta-glucosidase, an endoglucanase and a xylanase. In a preferred embodiment, the yield enhancing composition comprises a cellobiohydrolase I, a beta-glucosidase, an endoglucanase I, and a xylanase. In an embodiment, the yield enhancing composition comprises a cellobiohydrolase I, a beta-glucosidase, an endoglucanase II, and a xylanase. In an embodiment, the yield enhancing composition comprises a cellobiohydrolase II, a beta-glucosidase, an endoglucanase I, and a xylanase. In an embodiment, the yield enhancing composition comprises a cellobiohydrolase I, a beta-glucosidase, an endoglucanase II, and a xylanase. In a preferred embodiment, the yield enhancing composition comprises a cellobiohydrolase I, cellobiohydrolase II, a beta-glucosidase, an endoglucanase I, and a xylanase. In a preferred embodiment, the yield enhancing composition comprises a cellobiohydrolase I, cellobiohydrolase II, a beta-glucosidase, an endoglucanase I, an endoglucanase II, and a xylanase. In an embodiment, the yield enhancing composition comprises a cellobiohydrolase I, cellobiohydrolase II, a beta-glucosidase, an endoglucanase II, and a xylanase. In a preferred embodiment, the yield enhancing composition comprises a cellobiohydrolase II, a beta-glucosidase, an endoglucanase I, and a xylanase. The preceding yield enhancing compositions comprising cellulases and xylanases may further include at least one, at least two, at least three, at least four, or at least five different microorganisms (e.g., microbial strains, bacterial strains, mixtures, etc.).

In a preferred embodiment, the yield enhancing composition comprises a cellulase, a hemicellulase, and a pectinase. In a preferred embodiment, the yield enhancing composition comprises a cellulase, a hemicellulase, and a pectinase, wherein the cellulase is selected from the group consisting of a cellobiohydrolase, an endoglucanase, and a beta-glucosidase, wherein the hemicellulase is selected from the group consisting of endoxylanase, a beta-xylosidase, a arabinofuranosidase a glucuronidase, an acetyl xylan esterase, a feruloyl esterase, a coumaroyl esterase, an alpha-galactosidase, a beta-galactosidase, a beta-mannanase and a beta-mannosidase, wherein the pectinase is selected from the group consisting of an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and a xylogalacturonase,

In a preferred embodiment, the yield enhancing composition comprises a cellulase, a hemicellulase, and a pectinase. In a preferred embodiment, the yield enhancing composition comprises a cellulase, a hemicellulase, and a pectinase, wherein the cellulase is selected from the group consisting of a cellobiohydrolase, an endoglucanase, and a beta-glucosidase, wherein the hemicellulase is selected from the group consisting of endoxylanase, a beta-xylosidase, a arabinofuranosidase a glucuronidase, an acetyl xylan esterase, a feruloyl esterase, a coumaroyl esterase, an alpha-galactosidase, a beta-galactosidase, a beta-mannanase and a beta-mannosidase, wherein the pectinase is selected from the group consisting of an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and a xylogalacturonase, and at least one, at least two, at least three, at least four, or at least five different microorganisms. In a preferred embodiment, the yield enhancing composition comprises a cellulase, a hemicellulase, and a pectinase. In a preferred embodiment, the yield enhancing composition comprises a cellulase, a hemicellulase, and a pectinase, wherein the cellulase is selected from the group consisting of a cellobiohydrolase, an endoglucanase, and a beta-glucosidase, wherein the hemicellulase is selected from the group consisting of endoxylanase, a beta-xylosidase, a arabinofuranosidase a glucuronidase, an acetyl xylan esterase, a feruloyl esterase, a coumaroyl esterase, an alpha-galactosidase, a beta-galactosidase, a beta-mannanase and a beta-mannosidase, wherein the pectinase is selected from the group consisting of an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and a xylogalacturonase, and at least one, at least two, at least three, at least four, or at least five different microbial strains. In a preferred embodiment, the yield enhancing composition comprises a cellulase, a hemicellulase, and a pectinase. In a preferred embodiment, the yield enhancing composition comprises a cellulase, a hemicellulase, and a pectinase, wherein the cellulase is selected from the group consisting of a cellobiohydrolase, an endoglucanase, and a beta-glucosidase, wherein the hemicellulase is selected from the group consisting of endoxylanase, a beta-xylosidase, a arabinofuranosidase a glucuronidase, an acetyl xylan esterase, a feruloyl esterase, a coumaroyl esterase, an alpha-galactosidase, a beta-galactosidase, a beta-mannanase and a beta-mannosidase, wherein the pectinase is selected from the group consisting of an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and a xylogalacturonase, and at least one, at least two, at least three, at least four, or at least five different fungal strains.

In a preferred embodiment, the yield enhancing composition comprises a beta-glucanase and a pectinase. In a preferred embodiment, the yield enhancing composition comprises a beta-glucanase and a pectinase, wherein the beta-glucanase is selected from the group consisting of a beta-(1,3)-glucanase, a beta-(1,4)-glucanase a beta-(1,3)(1,4)-glucanase, and a beta-(1,6)-glucanase, and wherein the pectinase is selected from the group consisting of an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and a xylogalacturonase, In a preferred embodiment, the yield enhancing composition comprises a beta-glucanase and a pectinase, wherein the beta-glucanase is selected from the group consisting of a beta-(1,3)-glucanase, a beta-(1,4)-glucanase a beta-(1,3)(1,4)-glucanase, and a beta-(1,6)-glucanase, and wherein the pectinase is selected from the group consisting of an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and a xylogalacturonase, and at least one, at least two, at least three, at least four, or at least five different microorganisms.

In a preferred embodiment, the yield enhancing composition comprises a beta-glucanase and a pectinase, wherein the beta-glucanase is selected from the group consisting of a beta-(1,3)-glucanase, a beta-(1,4)-glucanase a beta-(1,3)(1,4)-glucanase, and a beta-(1,6)-glucanase, and wherein the pectinase is selected from the group consisting of an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and a xylogalacturonase, and at least one, at least two, at least three, at least four, or at least five different microbial strains. In a preferred embodiment, the yield enhancing composition comprises a beta-glucanase and a pectinase, wherein the beta-glucanase is selected from the group consisting of a beta-(1,3)-glucanase, a beta-(1,4)-glucanase a beta-(1,3)(1,4)-glucanase, and a beta-(1,6)-glucanase, and wherein the pectinase is selected from the group consisting of an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and a xylogalacturonase, and at least one, at least two, at least three, at least four, or at least five different fungal strains.

In a preferred embodiment, the yield enhancing composition comprises a beta-glucanase and a pectinase, and at least one, at least two, at least three, at least four, or at least five different microorganisms. In a preferred embodiment, the yield enhancing composition comprises a beta-glucanase and a pectinase, and at least one, at least two, at least three, at least four, or at least five different microorganisms, wherein the beta-glucanase is selected from the group consisting of a beta-(1,3)-glucanase, a beta-(1,4)-glucanase a beta-(1,3)(1,4)-glucanase, and a beta-(1,6)-glucanase, and wherein the pectinase is selected from the group consisting of an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and a xylogalacturonase, In a preferred embodiment, the yield enhancing composition comprises a beta-glucanase and a pectinase, and at least one, at least two, at least three, at least four, or at least five different microbial strains, wherein the beta-glucanase is selected from the group consisting of a beta-(1,3)-glucanase, a beta-(1,4)-glucanase a beta-(1,3)(1,4)-glucanase, and a beta-(1,6)-glucanase, and wherein the pectinase is selected from the group consisting of an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and a xylogalacturonase, In a preferred embodiment, the yield enhancing composition comprises a beta-glucanase and a pectinase, and at least one, at least two, at least three, at least four, or at least five different fungal strains, wherein the beta-glucanase is selected from the group consisting of a beta-(1,3)-glucanase, a beta-(1,4)-glucanase a beta-(1,3)(1,4)-glucanase, and a beta-(1,6)-glucanase, and wherein the pectinase is selected from the group consisting of an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and a xylogalacturonase,

In a preferred embodiment, the yield enhancing composition comprises a cellulase, xylanase, protease and lipase. In a preferred embodiment, the yield enhancing composition comprises a cellulase, xylanase, protease and lipase, and at least one, at least two, at least three, at least four, or at least five different microorganisms. In a preferred embodiment, the yield enhancing composition comprises a cellulase, xylanase, protease and lipase, and at least one, at least two, at least three, at least four, or at least five different microbial strains. In a preferred embodiment, the yield enhancing composition comprises a cellulase, xylanase, protease and lipase, and at least one, at least two, at least three, at least four, or at least five different fungal strains.

In a preferred embodiment, the yield enhancing composition comprises a cellulase, amylase, protease and lipase. In a preferred embodiment, the yield enhancing composition comprises a cellulase, amylase, protease and lipase, and at least one, at least two, at least three, at least four, or at least five different microorganisms. In a preferred embodiment, the yield enhancing composition comprises a cellulase, amylase, protease and lipase, and at least one, at least two, at least three, at least four, or at least five different microbial strains. In a preferred embodiment, the yield enhancing composition comprises a cellulase, amylase, protease and lipase, and at least one, at least two, at least three, at least four, or at least five different fungal strains.

In a preferred embodiment, the yield enhancing composition comprises a cellulase, alpha-amylase, protease and triacylglycerol lipase. In a preferred embodiment, the yield enhancing composition comprises a cellulase, alpha-amylase, protease and triacylglycerol lipase, and at least one, at least two, at least three, at least four, or at least five different microorganisms. In a preferred embodiment, the yield enhancing composition comprises a cellulase, alpha-amylase, protease and triacylglycerol lipase, and at least one, at least two, at least three, at least four, or at least five different microbial strains. In a preferred embodiment, the yield enhancing composition comprises a cellulase, alpha-amylase, protease and triacylglycerol lipase, and at least one, at least two, at least three, at least four, or at least five different fungal strains.

In a preferred embodiment, the yield enhancing composition comprises at least one, at least two, at least three cellulases selected from the group consisting of beta-glucosidase, cellobiohydrolase, and endoglucanase, an alpha-amylase, a protease and a triacylglycerol lipase. In a preferred embodiment, the yield enhancing composition at least one, at least two, at least three cellulases selected from the group consisting of beta-glucosidase, cellobiohydrolase, and endoglucanase, an alpha-amylase, a protease and a triacylglycerol lipase, and at least one, at least two, at least three, at least four, or at least five different microorganisms. In a preferred embodiment, the yield enhancing composition comprises at least one, at least two, at least three cellulases selected from the group consisting of beta-glucosidase, cellobiohydrolase, and endoglucanase, an alpha-amylase, a protease and a triacylglycerol lipase, and at least one, at least two, at least three, at least four, or at least five different microbial strains. In a preferred embodiment, the yield enhancing composition comprises at least one, at least two, at least three cellulases selected from the group consisting of beta-glucosidase, cellobiohydrolase, and endoglucanase, an alpha-amylase, a protease and a triacylglycerol lipase, and at least one, at least two, at least three, at least four, or at least five different fungal strains.

In a preferred embodiment, the yield enhancing composition comprises at least one, at least two, at least three, at least four, or at least five cellulases selected from the group consisting of beta-glucosidase, cellobiohydrolase I, cellobiohydrolase II, endoglucanase I, and endoglucanase II, an alpha-amylase, a protease and a triacylglycerol lipase. In a preferred embodiment, the yield enhancing composition at least one, at least two, at least three, at least four, or at least five cellulases selected from the group consisting of beta-glucosidase, cellobiohydrolase I, cellobiohydrolase II, endoglucanase I, and endoglucanase II, an alpha-amylase, a protease and a triacylglycerol lipase, and at least one, at least two, at least three, at least four, or at least five different microorganisms. In a preferred embodiment, the yield enhancing composition comprises at least one, at least two, at least three, at least four, or at least five cellulases selected from the group consisting of beta-glucosidase, cellobiohydrolase I, cellobiohydrolase II, endoglucanase I, and endoglucanase II, an alpha-amylase, a protease and a triacylglycerol lipase, and at least one, at least two, at least three, at least four, or at least five different microbial strains. In a preferred embodiment, the yield enhancing composition comprises at least one, at least two, at least three, at least four, or at least five cellulases selected from the group consisting of beta-glucosidase, cellobiohydrolase I, cellobiohydrolase II, endoglucanase I, and endoglucanase II, an alpha-amylase, a protease and a triacylglycerol lipase, and at least one, at least two, at least three, at least four, or at least five different fungal strains.

In a preferred embodiment, the yield enhancing composition comprises an endoglucanase (EG), a cellobiohydrolase (CBH), an endoxylanase (EX), a beta-xylosidase (BX) and a beta-glucosidase (BG). The xylanase may be a GH5 xylanase, a GH10 xylanase, or a GH11 xylanase. The endoglucanase may be a GH5 endoglucanase, a GH7 endoglucanase, or a mixture of a GH5 endoglucanase and a GH7 endoglucanase. The beta-xylosidase may be a GH3 beta-xylosidase. The cellobiohydrolase may be a CBHI or a CHBII.

In a preferred embodiment, the yield enhancing composition comprises at least one, at least two, at least three, at least four, at least five, at least six, or at least seven different enzyme types selected from the group consisting of an amylase, a beta-glucanases, a cellulase, a hemicellulase, a lipase, a pectinase, and a protease. In a preferred embodiment, the yield enhancing composition comprises at least one, at least two, at least three, at least four, at least five, at least six, or at least seven different enzyme types selected from the group consisting of an amylase, a beta-glucanases, a cellulase, a hemicellulase, a lipase, a pectinase, and a protease, and at least one, at least two, at least three, at least four, or at least five different microorganisms. In a preferred embodiment, the yield enhancing composition comprises at least one, at least two, at least three, at least four, at least five, at least six, or at least seven different enzyme types selected from the group consisting of an amylase, a beta-glucanases, a cellulase, a hemicellulase, a lipase, a pectinase, and a protease, and at least one, at least two, at least three, at least four, or at least five different microbial strains. In a preferred embodiment, the yield enhancing composition comprises at least one, at least two, at least three, at least four, at least five, at least six, or at least seven different enzyme types selected from the group consisting of an amylase, a beta-glucanases, a cellulase, a hemicellulase, a lipase, a pectinase, and a protease, and at least one, at least two, at least three, at least four, or at least five different fungal strains. The amylase may be an alpha-amylase, a glucoamylase, or a combination of alpha-amylase and glucoamylase.

In a preferred embodiment, the yield enhancing composition comprises an alpha-amylase and a glucoamylase. In a preferred embodiment, the yield enhancing composition comprises an alpha-amylase and a glucoamylase and at least one, at least two, at least three, at least four, or at least five different microorganisms. In a preferred embodiment, the yield enhancing composition comprises an alpha-amylase and a glucoamylase and at least one, at least two, at least three, at least four, or at least five different microbial strains. In a preferred embodiment, the yield enhancing composition comprises an alpha-amylase and a glucoamylase and at least one, at least two, at least three, at least four, or at least five different fungal strains.

In a preferred embodiment, the yield enhancing composition comprises a protease. In a preferred embodiment, the yield enhancing composition comprises a protease and at least one, at least two, at least three, at least four, or at least five different microorganisms. In a preferred embodiment, the yield enhancing composition comprises a protease and at least one, at least two, at least three, at least four, or at least five different microbial strains. In a preferred embodiment, the yield enhancing composition comprises a protease and at least one, at least two, at least three, at least four, or at least five different fungal strains.

The yield enhancing composition may include at least one, least two, at least three, at least four, or at least five different types of microorganisms. The microorganisms may be fungal strains, yeast strains, bacterial strains, or archaeal strains.

In an embodiment, the yield enhancing composition comprises at least one bacterial strain. In an embodiment, the yield enhancing composition comprises at least two bacterial strains. In an embodiment, the yield enhancing composition comprises at least three bacterial strains. In an embodiment, the yield enhancing composition comprises at least four bacterial strains. In an embodiment, the yield enhancing composition comprises at least five bacterial strains.

Examples of suitable bacterial strains include strains from the genera Bacillus, Pseudomonas, Acetobacter, Gluconobacter, Lactococcus, Lactobacillus, Pediococcus, Streptococcus, Aerococcus spp, Leuconostoc spp, Enterococcus and Propionibacterium.

In an embodiment, the at least one bacterial strain comprises a lactic acid bacterial strain. Suitable lactic acid bacterial strains include, but are not limited to, Lactococcus lactis ssp. lactis ATCC 19435, Lactococcus lactis ssp./lactis AS211, Lactobacillus delbrueckii ssp. delbrueckii ATCC 9649, Lactobacillus delbrueckii ssp. bulgaricus DSM 20081, Streptococcus salivarius subsp. thermophilus, Lactobacillus helveticus (thermophilic), Lactobacillus acidophilus, Lactobacillus bulgaricus, Pediococcus acidilactici, Streptococcus thermophilus, Streptococcus spp., Enterococcus spp, Pediococcus spp, Aerococcus spp, Leuconostoc spp, Lactobacillus salivarius, Lactobacillus brevis such as Lactobacillus brevis (DSM 23231), Lactobacillus fermentum, Lactobacillus reuteri, Lactobacillus hilgardii, Lactobacillus coryniformis, Lactobacillus curvatus, Lactobacillus plantarum such as Lactobacillus plantarum (DSM 19457), Lactobacillus casei, Lactobacillus curvatus, Leuconostoc mesenteroides sub-sp. cremoris, Lactococcus lactis, Enterococcus faecium, Lactobacillus kefir such as Lactobacillus kefir (DSM 19455), Lactobacillus buchneri, Lactobacillus paracasei, Lactobacillus diovilorans, Pediococcus pentosaceus, Lactobacillus rhamnosus, Pediococcus parvulus and Propionibacterium acidipropionici. In an embodiment the bacterial culture comprises the commercially available product called BioStabil® Biogas.

In an embodiment, the at least one bacterial strain is heterofermentative. The term “heterofermentative” as used herein means that the bacterial strain is capable of fermenting glucose into a variety of organic acids, like lactic acid, acetic acid, formic acid, citric acid, succinic acid and carbon dioxide. Heterofermentative bacterial strains include, but are not limited to, Leuconostoc spp, Lactobacillus salivarius, Lactobacillus brevis, Lactobacillus fermentum, Lactobacillus reuteri, Lactobacillus hilgardii, Lactobacillus coryniformis, Lactobacillus curvatus, Lactobacillus plantarum, Lactobacillus casei, Lactobacillus curvatus, Oenococcus, Weissella or any of the other bacterial cultures as described herein that are “heterofermentative”.

In a preferred embodiment, the yield enhancing composition comprises at least one heterofermentative bacterial strain. In a preferred embodiment, the yield enhancing composition comprises at least one heterofermentative bacterial strain and at least one non-heterofermentative bacterial strain.

In an embodiment, the yield enhancing composition comprises at least one, at least two, at least three, at least four, at least five, at least six, at least seven, at least eight, at least nine, or at least 10 different types of enzymes selected from the group consisting an amylase, cellulases, such as a cellobiohydrolase, an endo-p-1,4-glucanase, and/or a beta-glucosidase, beta-glucanases, such as a beta-(1,3)-glucanase, a beta-(1,4)-glucanase a beta-(1,3)(1,4)-glucanase, and/or a beta-(1,6)-glucanase, hemicellulases, for example, an endoxylanase, a beta-xylosidase, a arabinofuranosidase (e.g., alpha-L-arabionofuranosidase), a glucuronidase (e.g., an alpha-D-glucuronidase), an acetyl xylan esterase, a feruloyl esterase, a coumaroyl esterase, an alpha-galactosidase, a beta-galactosidase, a beta-mannanase and/or a beta-mannosidase, a lytic polysaccharide monooxygenase (e.g. GH61), pectinases, for example, an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and/or a xylogalacturonase, a protease, a lipase, a phytase, a ligninase, a hexosyltransferase, a glucuronidase, an expansin, a cellulose induced protein or a cellulose integrating protein or like protein, a lactase, a sucrase, a catalase, and at least one, at least two, at least three, at least four, or at least five different types of bacterial strains selected from the genera Bacillus, Pseudomonas, Acetobacter, Gluconobacter, Lactococcus, Lactobacillus, Pediococcus, Streptococcus, Aerococcus spp, Leuconostoc spp, Enterococcus and Propionibacterium.

In an embodiment, the yield enhancing composition comprises at least one bacterial strain of the genus Bacillus. Examples of suitable Bacillus strains include Bacillus amyloliquefaciens, Bacillus megaterium, Bacillus pumilus, Bacillus subtilis, and/or Bacillus thuringiensis.

An exemplary Pseudomonas strain is Pseudomonas monteilii. In an embodiment, the bacterial strain is a Bacillus spp. strain. In an embodiment, the bacterial strain is a Pseudomonas spp. strain. In an embodiment, the bacterial strains comprise a Bacillus spp. strain and a Pseudomonas spp. strain. In an embodiment, the bacterial strain is Bacillus amyloliquefaciens. In an embodiment, the bacterial strain is Bacillus megaterium. In an embodiment, the bacterial strain is Bacillus subtilis. In an embodiment, the bacterial strain is Bacillus thuringiensis. In an embodiment, the bacterial strains comprise Bacillus amyloliquefaciens and Bacillus megaterium strains. In an embodiment, the bacterial strains comprise Bacillus amyloliquefaciens and Bacillus subtilis strains. In an embodiment, the bacterial strains comprise Bacillus amyloliquefaciens and Bacillus thuringiensis strains. In an embodiment, the bacterial strains comprises Bacillus amyloliquefaciens, Bacillus megaterium and Bacillus subtilis strains. In an embodiment, the bacterial strains comprise Bacillus amyloliquefaciens, Bacillus megaterium and Bacillus thuringiensis strains. In an embodiment, the bacterial strains comprise Bacillus amyloliquefaciens, Bacillus subtilis and Bacillus thuringiensis strains. In an embodiment, the bacterial strains comprise Bacillus amyloliquefaciens, Bacillus megaterium, Bacillus subtilis and Bacillus thuringiensis strains. In an embodiment, the bacterial strain is Pseudomonas monteilii. In an embodiment, the bacterial strains comprise Bacillus amyloliquefaciens, Bacillus megaterium, Bacillus subtilis, and Pseudomonas monteilii strains. In an embodiment, the bacterial strains comprise Bacillus amyloliquefaciens, Bacillus megaterium, Bacillus thuringiensis, and Pseudomonas monteilii strains. In an embodiment, the bacterial strains comprise Bacillus amyloliquefaciens, Bacillus subtilis, Bacillus thuringiensis, and Pseudomonas monteilii strains. In an embodiment, the bacterial strains comprise Bacillus amyloliquefaciens, Bacillus megaterium, Bacillus subtilis, Bacillus thuringiensis, and Pseudomonas monteilii strains.

In a preferred embodiment, the yield enhancing composition comprises a cellulase, amylase, protease and lipase, and at least one bacterial strain selected from the group consisting of Bacillus amyloliquefaciens, Bacillus megaterium, Bacillus pumilus, Bacillus subtilis, Bacillus thuringiensis, and any combination thereof.

In a preferred embodiment, the yield enhancing composition comprises a cellulase, amylase, protease and lipase, and the bacterial strains Bacillus amyloliquefaciens, Bacillus megaterium, Bacillus pumilus, Bacillus subtilis, and Bacillus thuringiensis.

In a preferred embodiment, the yield enhancing composition comprises a cellulase, amylase, protease and lipase, and at least one bacterial strain of the genus Pseudomonas.

In a preferred embodiment, the yield enhancing composition comprises a cellulase, amylase, protease and lipase, and Pseudomonas monteilii.

In a preferred embodiment, the yield enhancing composition comprises a cellulase, amylase, protease and lipase, and bacterial strains of the genera Bacillus and Pseudomonas.

In a preferred embodiment, the yield enhancing composition comprises a cellulase, amylase, protease and lipase, and Bacillus amyloliquefaciens, Bacillus megaterium, Bacillus pumilus, Bacillus subtilis, Bacillus thuringiensis, and Pseudomonas monteilii.

In a preferred embodiment, the yield enhancing composition comprises a cellulase, alpha-amylase, protease a triacylglycerol lipase, and at least one bacterial strain selected from the group consisting of Bacillus amyloliquefaciens, Bacillus megaterium, Bacillus pumilus, Bacillus subtilis, Bacillus thuringiensis, and any combination thereof.

In a preferred embodiment, the yield enhancing composition comprises a cellulase, alpha-amylase, protease a triacylglycerol lipase, and Bacillus amyloliquefaciens, Bacillus megaterium, Bacillus pumilus, Bacillus subtilis, and Bacillus thuringiensis.

In a preferred embodiment, the yield enhancing composition comprises a cellulase, alpha-amylase, protease a triacylglycerol lipase, and at least one bacterial strain of the genus Pseudomonas.

In a preferred embodiment, the yield enhancing composition comprises a cellulase, alpha-amylase, protease a triacylglycerol lipase, and Pseudomonas monteilii.

In a preferred embodiment, the yield enhancing composition comprises a cellulase, alpha-amylase, protease a triacylglycerol lipase, and bacterial strains of the genera Bacillus and Pseudomonas.

In a preferred embodiment, the yield enhancing composition comprises a cellulase, alpha-amylase, protease a triacylglycerol lipase, and Bacillus amyloliquefaciens, Bacillus megaterium, Bacillus pumilus, Bacillus subtilis, Bacillus thuringiensis, and Pseudomonas monteilii.

In a preferred embodiment, the yield enhancing composition comprises at least one, at least two, at least three cellulases selected from the group consisting of beta-glucosidase, cellobiohydrolase, and endoglucanase, an alpha-amylase, a protease and a triacylglycerol lipase, and at least one bacterial strain selected from the group consisting of Bacillus amyloliquefaciens, Bacillus megaterium, Bacillus pumilus, Bacillus subtilis, Bacillus thuringiensis, and any combination thereof.

In a preferred embodiment, the yield enhancing composition comprises at least one, at least two, at least three cellulases selected from the group consisting of beta-glucosidase, cellobiohydrolase, and endoglucanase, an alpha-amylase, a protease and a triacylglycerol lipase, and Bacillus amyloliquefaciens, Bacillus megaterium, Bacillus pumilus, Bacillus subtilis, and Bacillus thuringiensis.

In a preferred embodiment, the yield enhancing composition comprises at least one, at least two, at least three cellulases selected from the group consisting of beta-glucosidase, cellobiohydrolase, and endoglucanase, an alpha-amylase, a protease and a triacylglycerol lipase, and at least one bacterial strain of the genus Pseudomonas.

In a preferred embodiment, the yield enhancing composition comprises at least one, at least two, at least three cellulases selected from the group consisting of beta-glucosidase, cellobiohydrolase, and endoglucanase, an alpha-amylase, a protease and a triacylglycerol lipase, and Pseudomonas monteilii.

In a preferred embodiment, the yield enhancing composition comprises at least one, at least two, at least three cellulases selected from the group consisting of beta-glucosidase, cellobiohydrolase, and endoglucanase, an alpha-amylase, a protease and a triacylglycerol lipase, and bacterial strains of the genera Bacillus and Pseudomonas.

In a preferred embodiment, the yield enhancing composition comprises at least one, at least two, at least three cellulases selected from the group consisting of beta-glucosidase, cellobiohydrolase, and endoglucanase, an alpha-amylase, a protease and a triacylglycerol lipase, and Bacillus amyloliquefaciens, Bacillus megaterium, Bacillus pumilus, Bacillus subtilis, Bacillus thuringiensis, and Pseudomonas monteilii.

In a preferred embodiment, the yield enhancing composition comprises at least one, at least two, at least three, at least four, or at least five cellulases selected from the group consisting of beta-glucosidase, cellobiohydrolase I, cellobiohydrolase II, endoglucanase I, and endoglucanase II, an alpha-amylase, a protease and a triacylglycerol lipase, and at least one bacterial strain selected from the group consisting of Bacillus amyloliquefaciens, Bacillus megaterium, Bacillus pumilus, Bacillus subtilis, Bacillus thuringiensis, and any combination thereof.

In a preferred embodiment, the yield enhancing composition comprises at least one, at least two, at least three, at least four, or at least five cellulases selected from the group consisting of beta-glucosidase, cellobiohydrolase I, cellobiohydrolase II, endoglucanase I, and endoglucanase II, an alpha-amylase, a protease and a triacylglycerol lipase, and Bacillus amyloliquefaciens, Bacillus megaterium, Bacillus pumilus, Bacillus subtilis, and Bacillus thuringiensis.

In a preferred embodiment, the yield enhancing composition comprises at least one, at least two, at least three, at least four, or at least five cellulases selected from the group consisting of beta-glucosidase, cellobiohydrolase I, cellobiohydrolase II, endoglucanase I, and endoglucanase II, an alpha-amylase, a protease and a triacylglycerol lipase, and at least one bacterial strain of the genus Pseudomonas.

In a preferred embodiment, the yield enhancing composition comprises at least one, at least two, at least three, at least four, or at least five cellulases selected from the group consisting of beta-glucosidase, cellobiohydrolase I, cellobiohydrolase II, endoglucanase I, and endoglucanase II, an alpha-amylase, a protease and a triacylglycerol lipase, and Pseudomonas monteilii.

In a preferred embodiment, the yield enhancing composition comprises at least one, at least two, at least three, at least four, or at least five cellulases selected from the group consisting of beta-glucosidase, cellobiohydrolase I, cellobiohydrolase II, endoglucanase I, and endoglucanase II, an alpha-amylase, a protease and a triacylglycerol lipase, and bacterial strains of the genera Bacillus and Pseudomonas.

In a preferred embodiment, the yield enhancing composition comprises at least one, at least two, at least three, at least four, or at least five cellulases selected from the group consisting of beta-glucosidase, cellobiohydrolase I, cellobiohydrolase II, endoglucanase I, and endoglucanase II, an alpha-amylase, a protease and a triacylglycerol lipase, and Bacillus amyloliquefaciens, Bacillus megaterium, Bacillus pumilus, Bacillus subtilis, Bacillus thuringiensis, and Pseudomonas monteilii.

In a preferred embodiment, the yield enhancing composition comprises a cellulase, a hemicellulase, Bacillus amyloliquefaciens, Bacillus megaterium, Bacillus pumilus, Bacillus subtilis, Bacillus thuringiensis, and Pseudomonas monteilii.

In a preferred embodiment, the yield enhancing composition comprises a cellulase, a xylanase, Bacillus amyloliquefaciens, Bacillus megaterium, Bacillus pumilus, Bacillus subtilis, Bacillus thuringiensis, and Pseudomonas monteilii.

In a preferred embodiment, the yield enhancing composition comprises a cellulase, a xylanase, a protease, a lipase, Bacillus amyloliquefaciens, Bacillus megaterium, Bacillus pumilus, Bacillus subtilis, Bacillus thuringiensis, and Pseudomonas monteilii.

In a preferred embodiment, the yield enhancing composition comprises a cellulase, a xylanase, a protease, a triacylglycerol lipase, Bacillus amyloliquefaciens, Bacillus megaterium, Bacillus pumilus, Bacillus subtilis, Bacillus thuringiensis, and Pseudomonas monteilii.

In an embodiment, the yield enhancing composition comprises at least one fungal strain.

Examples of suitable fungal strains include strains of the genera Acremonium, Agahcus, Aspergillus, Aureobasidium, Beauvaria, Cephalosporium, Ceriporiopsis, Chaetomium paecilomyces, Chrysosporium, Claviceps, Cochiobolus, Coprinus, Cryptococcus, Cyathus, Emericella, Endothia, Endothia mucor, Filibasidium, Fusarium, Geosmithia, Gilocladium, Humicola, Magnaporthe, Mucor, Myceliophthora, Myrothecium, Neocallimastix, Neurospora, Paecilomyces, Penicillium, Piromyces, Panerochaete, Pleurotus, Podospora, Pyricularia, Rasamsonia, Rhizomucor, Rhizopus, Scylatidium, Schizophyllum, Stagonospora, Talaromyces, Thermoascus, Thermomyces, Thielavia, Tolypocladium, Trametes pleurotus, Trichoderma and Trichophyton.

In an embodiment, the yield enhancing composition comprises at least two, at least three, at least four, at least five, at least six, at least seven, at least eight, at least nine, or at least 10 different types of enzymes selected from the group consisting an amylase, cellulases, such as a cellobiohydrolase, an endo-p-1,4-glucanase, and/or a beta-glucosidase, beta-glucanases, such as a beta-(1,3)-glucanase, a beta-(1,4)-glucanase a beta-(1,3)(1,4)-glucanase, and/or a beta-(1,6)-glucanase, hemicellulases, for example, an endoxylanase, a beta-xylosidase, a arabinofuranosidase (e.g., alpha-L-arabionofuranosidase), a glucuronidase (e.g., an alpha-D-glucuronidase), an acetyl xylan esterase, a feruloyl esterase, a coumaroyl esterase, an alpha-galactosidase, a beta-galactosidase, a beta-mannanase and/or a beta-mannosidase, a lytic polysaccharide monooxygenase (e.g. GH61), pectinases, for example, an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and/or a xylogalacturonase, a protease, a lipase, a phytase, a ligninase, a hexosyltransferase, a glucuronidase, an expansin, a cellulose induced protein or a cellulose integrating protein or like protein, a lactase, a sucrase, a catalase, and at least one, at least two, at least three, at least four, or at least five fungal strains of the genera Acremonium, Agahcus, Aspergillus, Aureobasidium, Beauvaria, Cephalosporium, Ceriporiopsis, Chaetomium paecilomyces, Chrysosporium, Claviceps, Cochiobolus, Coprinus, Cryptococcus, Cyathus, Emericella, Endothia, Endothia mucor, Filibasidium, Fusarium, Geosmithia, Gilocladium, Humicola, Magnaporthe, Mucor, Myceliophthora, Myrothecium, Neocallimastix, Neurospora, Paecilomyces, Penicillium, Piromyces, Panerochaete, Pleurotus, Podospora, Pyricularia, Rasamsonia, Rhizomucor, Rhizopus, Scylatidium, Schizophyllum, Stagonospora, Talaromyces, Thermoascus, Thermomyces, Thielavia, Tolypocladium, Trametes pleurotus, Trichoderma and Trichophyton.

Examples of suitable Aspergillus strains include, but are not limited to, Aspergillus niger, Aspergillus flavus, Aspergillus ustus, and Aspergillus wentii.

Examples of suitable Rhizopus fungal strains include, but are not limited to, Rhizopus arrhizus and Rhizopus oryzae.

Examples of suitable Trichodmera fungal strains include, but are not limited to, Trichoderma inhamatum, Trichoderma reesei, and Talaromyces emersonii.

In an embodiment, the yeast enhancing composition comprises at least one yeast strain. Examples of suitable yeast strains include, but are not limited to, Candida lignohabitans, Candida catenula, Candida guilliermondii, Yarrowia lipolytica, Candida tropicalis, Kluyveromyces, and Saccharomyces.

In an embodiment, the yield enhancing composition comprises at least two, at least three, at least four, at least five, at least six, at least seven, at least eight, at least nine, or at least 10 different types of enzymes selected from the group consisting an amylase, cellulases, such as a cellobiohydrolase, an endo-p-1,4-glucanase, and/or a beta-glucosidase, beta-glucanases, such as a beta-(1,3)-glucanase, a beta-(1,4)-glucanase a beta-(1,3)(1,4)-glucanase, and/or a beta-(1,6)-glucanase, hemicellulases, for example, an endoxylanase, a beta-xylosidase, a arabinofuranosidase (e.g., alpha-L-arabionofuranosidase), a glucuronidase (e.g., an alpha-D-glucuronidase), an acetyl xylan esterase, a feruloyl esterase, a coumaroyl esterase, an alpha-galactosidase, a beta-galactosidase, a beta-mannanase and/or a beta-mannosidase, a lytic polysaccharide monooxygenase (e.g. GH61), pectinases, for example, an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and/or a xylogalacturonase, a protease, a lipase, a phytase, a ligninase, a hexosyltransferase, a glucuronidase, an expansin, a cellulose induced protein or a cellulose integrating protein or like protein, a lactase, a sucrase, a catalase, and at least one, at least two, at least three, at least four, or at least five yeast strains selected from Candida lignohabitans, Candida catenula, Candida guilliermondii, Yarrowia lipolytica, Candida tropicalis, Kluyveromyces, and Saccharomyces.

An exemplary Mucor strain is Mucor racemosus. An exemplary Paecilomyces strain is Paecilomyces lilacinus. An exemplary Trichoderma strain is Trichoderma inhamatum.

In an aspect, the fungal strain comprises an Aspergillus spp. strain. In an embodiment, the fungal strain comprises a Mucor spp. strain. In an embodiment, the fungal strain comprises a Paecilomyces spp. strain. In an embodiment, the fungal strain comprises a Trichoderma spp. strain. In an embodiment, the fungal strains comprise Aspergillus spp. and Mucor spp. strains. In an embodiment, the fungal strains comprise Aspergillus spp. and Paecilomyces spp. strains. In an embodiment, the fungal strains comprise Aspergillus spp. and Trichoderma spp. strains. In an embodiment, the fungal strain comprise Mucor spp. and Paecilomyces spp. strains. In an embodiment, the fungal strains comprise Mucor spp. and Trichoderma spp. strains. In an embodiment, the fungal strains comprise Paecilomyces spp. and Trichoderma spp. strains. In an embodiment, the fungal strain comprise Paecilomyces spp. and Trichoderma spp. strains. In an embodiment, the fungal strains comprise Mucor spp., Paecilomyces spp., and Trichoderma spp. strains. In an embodiment, the fungal strains comprise Aspergillus spp., Mucor spp., and Paecilomyces spp. strains. In an embodiment, the fungal strains comprise Aspergillus spp., Mucor spp., and Trichoderma spp. strains. In an embodiment, the fungal strains comprise Aspergillus spp., Mucor spp., Paecilomyces spp., and Trichoderma spp. strains. In an embodiment, the fungal strain is Aspergillus ustus. In an embodiment, the fungal strain is Mucor racemosus. In an embodiment, the fungal strain is Paecilomyces lilacinus. In an embodiment, the fungal strain is Trichoderma inhamatum. In an embodiment, the fungal strains comprise Mucor racemosus and Paecilomyces lilacinus strains. In an embodiment, the fungal strains comprise Mucor racemosus and Aspergillus ustus strains. In an embodiment, the fungal strains comprise Mucor racemosus and Trichoderma inhamatum strains. In an embodiment, the fungal strains comprise Paecilomyces lilacinus and Aspergillus ustus strains. In an embodiment, the fungal strains comprise Paecilomyces lilacinus and Trichoderma inhamatum strains. In an embodiment, the fungal strains comprise Aspergillus ustus and Trichoderma inhamatum strains. In an embodiment, the fungal strains comprise Mucor racemosus, Paecilomyces lilacinus, and Aspergillus ustus strains. In an embodiment, the fungal strains comprise Mucor racemosus, Paecilomyces lilacinus, and Trichoderma inhamatum strains. In an embodiment, the fungal strains comprise Paecilomyces lilacinus, Aspergillus ustus and Trichoderma inhamatum strains. In an embodiment, the fungal strains comprise Mucor racemosus, Paecilomyces lilacinus, Aspergillus ustus and Trichoderma inhamatum strains.

In an embodiment, the yield enhancing composition comprises at least one fungal strain and at least one bacterial strain. In an embodiment, the yield enhancing composition comprises at least two fungal strains and at least one bacterial strain. In an embodiment, the yield enhancing composition comprises at least three fungal strains and at least one bacterial strain. In an embodiment, the yield enhancing composition comprises at least four fungal strains and at least one bacterial strain. In an embodiment, the yield enhancing composition comprises at least one fungal strain and at least two bacterial strains. In an embodiment, the yield enhancing composition comprises at least one fungal strain and at least three bacterial strains. In an embodiment, the yield enhancing composition comprises at least one fungal strain and at least four bacterial strains. In an embodiment, the yield enhancing composition comprises at least two fungal strains and at least two bacterial strains. In an embodiment, the yield enhancing composition comprises at least three fungal strains and at least two bacterial strains. In an embodiment, the yield enhancing composition comprises at least four fungal strains and at least two bacterial strains. In an embodiment, the yield enhancing composition comprises at least five fungal strains and at least two bacterial strains. In an embodiment, the yield enhancing composition comprises at least three fungal strains and at least two bacterial strains. In an embodiment, the yield enhancing composition comprises at least two fungal strains and at least three bacterial strains. In an embodiment, the yield enhancing composition comprises at least two fungal strains and at least four bacterial strains. In an embodiment, the yield enhancing composition comprises at least two fungal strains and at least five bacterial strains. In an embodiment, the yield enhancing composition comprises at least three fungal strains and at least three bacterial strains. In an embodiment, the yield enhancing composition comprises at least four fungal strains and at least three bacterial strains. In an embodiment, the yield enhancing composition comprises at least five fungal strains and at least three bacterial strains. In an embodiment, the yield enhancing composition comprises at least three fungal strains and at least four bacterial strains. In an embodiment, the yield enhancing composition comprises at least three fungal strains and at least five bacterial strains. In an embodiment, the yield enhancing composition comprises at least four fungal strains and at least four bacterial strains. In an embodiment, the yield enhancing composition comprises at least five fungal strains and at least four bacterial strains. In an embodiment, the yield enhancing composition comprises at least four fungal strains and at least five bacterial strains. In an embodiment, the yield enhancing composition comprises at least five fungal strains and at five three bacterial strains.

In an embodiment, the yield enhancing composition comprises at least one fungal strain, at least one yeast strain, and at least one bacterial strain. In an embodiment, the yield enhancing composition comprises at least two, at least three, at least four, at least five, at least six, at least seven, at least eight, at least nine, or at least 10 different types of enzymes selected from the group consisting an amylase, cellulases, such as a cellobiohydrolase, an endo-p-1,4-glucanase, and/or a beta-glucosidase, beta-glucanases, such as a beta-(1,3)-glucanase, a beta-(1,4)-glucanase a beta-(1,3)(1,4)-glucanase, and/or a beta-(1,6)-glucanase, hemicellulases, for example, an endoxylanase, a beta-xylosidase, a arabinofuranosidase (e.g., alpha-L-arabionofuranosidase), a glucuronidase (e.g., an alpha-D-glucuronidase), an acetyl xylan esterase, a feruloyl esterase, a coumaroyl esterase, an alpha-galactosidase, a beta-galactosidase, a beta-mannanase and/or a beta-mannosidase, a lytic polysaccharide monooxygenase (e.g. GH61), pectinases, for example, an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and/or a xylogalacturonase, a protease, a lipase, a phytase, a ligninase, a hexosyltransferase, a glucuronidase, an expansin, a cellulose induced protein or a cellulose integrating protein or like protein, a lactase, a sucrase, a catalase, and at least one, at least two, at least three, at least four, or at least five different microorganisms selected from the group consisting of yeast strains, fungal strains, and bacterial strains, wherein the yeast strains are selected from Candida lignohabitans, Candida catenula, Candida guilliermondii, Yarrowia lipolytica, Candida tropicalis, Kluyveromyces, and Saccharomyces, wherein the fungal strains are selected from Acremonium, Agahcus, Aspergillus, Aureobasidium, Beauvaria, Cephalosporium, Ceriporiopsis, Chaetomium paecilomyces, Chrysosporium, Claviceps, Cochiobolus, Coprinus, Cryptococcus, Cyathus, Emericella, Endothia, Endothia mucor, Filibasidium, Fusarium, Geosmithia, Gilocladium, Humicola, Magnaporthe, Mucor, Myceliophthora, Myrothecium, Neocallimastix, Neurospora, Paecilomyces, Penicillium, Piromyces, Panerochaete, Pleurotus, Podospora, Pyricularia, Rasamsonia, Rhizomucor, Rhizopus, Scylatidium, Schizophyllum, Stagonospora, Talaromyces, Thermoascus, Thermomyces, Thielavia, Tolypocladium, Trametes pleurotus, Trichoderma and Trichophyton, and wherein the bacterial strains are selected from Bacillus, Pseudomonas, Acetobacter, Gluconobacter, Lactococcus, Lactobacillus, Pediococcus, Streptococcus, Aerococcus spp, Leuconostoc spp, Enterococcus and Propionibacterium.

The invention is further summarized in the following paragraphs:
1. A method for carrying out the combined operation of a bioethanol production unit and a biogas unit, wherein:
   a) milled grain from a dry milling step is mashed with at least 0.1 t of dry matter in the form of whole stillage and at least 0.1 m³ of outflow from the biogas unit per tonne of milled grain,
   b) the mash from 1a) is fed to a cooking stage with mash temperatures below the gelatinization temperature of the starch in the milled grain, followed by an ethanol-forming fermentation step and then feeding the fermented mash to a distillation step to produce a whole stillage, the whole stillage from 1b) is fed to the mashing step in a) and to the biogas unit, wherein a yield enhancing composition comprising at least one, at least two, at least three, at least four, or at least five different types of enzymes and/or at least one, at least two, at least three, at least four, or at least five different microorganism strains is:
   i) added to the biogas unit; and/or
   ii) added to the whole stillage from 1b) that is fed to the mashing step in 1a); and/or
   iii) added to the whole stillage from 1b) that is fed to the biogas unit; and/or
   iv) added to the outflow of the biogas unit before being mashed with the corn meal from step 1a).
2. The method of paragraph 1, wherein at least 0.2 t TS per tonne of milled grain is added to the mashing step in 1a) in the form of a mixture of thin stillage and whole stillage.
3. The method of paragraph 1 or 2, wherein at least 0.12 t TS per tonne of milled grain is recycled to the mashing step in 1a) in the form of whole stillage.
4. The method of any one of paragraphs 1-3, wherein at least 0.1 t TS of thin stillage per t of mash is recycled to the mashing step in 1a).
5. The method of any one of paragraphs 1-4, wherein the cooking stage in 1b), the mash is heated to a maximum of 70 degrees centigrade.
6. The method of any one of paragraphs 1-5, wherein the milled grain in 1a) from a dry milling step has a mass fraction of at least 60 percent of particles with a particle size of <0.5 mm.
7. The method of any one of paragraphs 1-6, wherein the pH of the mash in 1a) is adjusted to less than 4.5.
8. The method of any one of paragraphs 1-7, wherein the ammonium nitrogen content in the biogas fermenters of the biogas unit is kept at between 6000-9000 ppm.
9. The method of any one of paragraphs 1-8, wherein the mean hydraulic residence time for the biogas unit is at least 30 days.
10. The method of any one of paragraphs 1-9, wherein the proportion of outflow from the biogas unit per tonne of milled grain in 1a) is at least 0.2 m³.
11. The method of any one of paragraphs 1-10, wherein the distillation step in 1b), at least 400 liters of ethanol are separated per tonne of milled grain.
12. The method of any one of paragraphs 1-11, wherein the mashing step in 1a), at least 100 g of ammonium nitrogen per tonne of milled grain is recycled via the outflow from the biogas unit.
13. The method of any one of paragraphs 1-12, wherein the mashing step in 1a), a maximum of 1000 g of ammonium nitrogen per tonne of milled grain is recycled via the outflow from the biogas unit.
14. The method of any one of paragraphs 1-13, wherein less than 1.2 m³ of fresh water is used per tonne of milled grain.
15. The method of any one of paragraphs 1-14, wherein at least 15 kg of glycerin per tonne of milled grain is recycled to the mashing step via the whole stillage and thin stillage.
16. The method of any one of paragraphs 1-15, wherein whole stillage from step b) is fed to a solid-liquid separation step to generate thin stillage and wet cake.
17. The method of any one of paragraphs 1-16, wherein the thin stillage is fed to the mashing step in 1a).
18. The method of any one of paragraphs 1-17, wherein the yield enhancing composition comprising at least two, at least three, at least four, or at least five different enzymes and/or at least one, at least two, at least three, at least four or at least five different microorganism strains are added to the thin stillage that is fed to the mashing step in 1a).
19. The method of any one of paragraphs 1-18, wherein the thin stillage is used to make corn oil and residual materials resulting from purification of the corn oil.
20. The method of any one of paragraphs 1-19, wherein the residual materials resulting from purification of the corn oil are fed to the biogas unit.
21. The method of any one of paragraphs 1-20, wherein the yield enhancing composition comprising at least two, at least three, at least four, or at least five different types of enzymes and/or at least one, at least two, at least three, at least four, or at least five different microorganism strains are added to the residual materials resulting from purification of the corn oil that are fed to the biogas unit.
22. The method of any one of paragraphs 1-21, wherein the thin stillage is used to produce a protein product with a raw protein content of more than 44 percent and residual materials resulting from purification of the protein product.
23. The method of any one of paragraphs 1-22, wherein the residual materials resulting from purification of the protein product are fed to the biogas unit.
24. The method of any one of paragraphs 1-23, wherein the yield enhancing composition comprising at least two, at least three, at least four, or at least five different types of enzymes and/or at least one, at least two, at least three, at least four, or at least five different microorganism strains are added to the residual materials resulting from purification of the protein product that are fed to the biogas unit.
25. The method of any one of paragraphs 1-24, wherein the wet cake is fed to the mashing in step 1a).
26. The method of any one of paragraphs 1-25, wherein the yield enhancing composition comprising at least two, at least three, at least four, or at least five different enzymes and/or at least one, at least two, at least three, at least four or at least five different microorganism strains are added to the wet cake that is fed to the mashing step in 1a).
27. The method of any one of paragraphs 1-26, wherein the wet cake is fed to the biogas unit.
28. The method of any one of paragraphs 1-27, wherein a yield enhancing composition comprising at least two, at least three, at least four, or at least five different enzymes and/or at least one, at least two, at least three, at least four, or at least five different microorganism strains is added to the wet cake that is fed to the biogas unit.
29. The method of any one of paragraphs 1-28, wherein the milled grain comprises corn meal.
30. The method as claimed in any one of claims 1-29, wherein a biomass other than the milled grain is fed to the biogas unit.
31. The method of any one of paragraphs 1-30, wherein the biomass comprises cellulosic material and/or lignocellulosic material and/or hemicellulosic material and/or starch material and/or pectin material and/or lipid material.
32. The method of any one of paragraphs 1-31, wherein the biomass comprises pretreated corn stover.
33. The method of any one of paragraphs 1-32, wherein the thin stillage is mixed with pig manure and the yield enhancing composition is added to the mixture and the mixture is fed to the biogas unit.
34. The method of any one of paragraphs 1-33, wherein the thin stillage is mixed with corn sillage and the yield enhancing composition is added to the mixture and the mixture is fed to the biogas unit.
35. The method of any one of paragraphs 1-34, wherein the thin stillage is mixed with food waste and the yield enhancing composition is added to the mixture and the mixture is fed to the biogas unit.
36. The method of any one of paragraphs 1-35, wherein the thin stillage is mixed with municipal solid waste and the yield enhancing composition is added to the mixture and the mixture is fed to the biogas unit.
37. The method of any one of paragraphs 1-36, wherein the thin stillage is mixed with cassava pulp and the yield enhancing composition is added to the mixture and the mixture is fed to the biogas unit.
38. The method of any one of paragraphs 1-37, wherein the thin stillage is mixed with sugar beet pulp and the yield enhancing composition is added to the mixture and the mixture is fed to the biogas unit.
39. The method of any one of paragraphs 1-38, wherein the thin stillage is mixed with grass clippings and the yield enhancing composition is added to the mixture and the mixture is fed to the biogas unit.
40. The method of any one of paragraphs 1-39, wherein the thin stillage is mixed with straw pellets and the yield enhancing composition is added to the mixture and the mixture is fed to the biogas unit.
41. The method of any one of paragraphs 1-40, wherein the thin stillage is mixed with pretreated corn stover and the yield enhancing composition is added to the mixture and the mixture is fed to the biogas unit.
42. The method of any one of paragraphs 1-41, wherein the yield enhancing composition comprises a cellulase.
43. The method of any one of paragraphs 1-42, wherein the yield enhancing composition comprises a beta-glucosidase.
44. The method of any one of paragraphs 1-43, wherein the yield enhancing composition comprises an endoglucanase.
45. The method of any one of paragraphs 1-44, wherein the yield enhancing composition comprises a cellobiohydrolase.
46. The method of any one of paragraphs 1-45, wherein the yield enhancing composition comprises an endoglucanase, a beta-glucosidase and a cellobiohydrolase.
47. The method of any one of paragraphs 1-46, wherein the yield enhancing composition comprises a beta-glucosidase, a cellobiohydrolase I, a cellobiohydrolase II, an Auxilary Activity Family 9 (AA9) polypeptide, a xylanase and a beta-xylosidase.
48. The method of any one of paragraphs 1-47, wherein the yield enhancing composition comprises a beta-glucosidase, a cellobiohydrolase I, a cellobiohydrolase II, an AA9 polypeptide, a xylanase, a beta-xylosidase, and a protease.
49. The method of any one of paragraphs 1-48, wherein the yield enhancing composition comprises a beta-glucosidase, a cellobiohydrolase, an endoglucanase, an arabinofuranosidase, and a xylanase
50. The method of any one of paragraphs 1-49, wherein the yield enhancing composition comprises a beta-glucosidase, a cellobiohydrolase, an endoglucanase, an arabinofuranosidase, a xylanase and a protease.
51. The method of any one of paragraphs 1-50, wherein the yield enhancing composition comprises a lipase.
52. The method of any one of paragraphs 1-51, wherein the yield enhancing composition comprises a protease.
53. The method of any one of paragraphs 1-52, wherein the yield enhancing composition comprises a cellulase and a xylanase.
54. The method of any one of paragraphs 1-53, wherein the yield enhancing composition comprises a beta-glucosidase and a xylanase.
55. The method of any one of paragraphs 1-54, wherein the yield enhancing composition comprises an endoglucanase and a xylanase.
56. The method of any one of paragraphs 1-55, wherein the yield enhancing composition comprises a cellobiohydrolase and a xylanase.
57. The method of any one of paragraphs 1-56, wherein the yield enhancing composition comprises an endoglucanase, a beta-glucosidase, a cellobiohydrolase and a xylanase.
58. The method of any one of paragraphs 1-57, wherein the yield enhancing composition comprises an amylase.
59. The method of any one of paragraphs 1-58, wherein the yield enhancing composition comprises an amylase and a glucoamylase.
60. The method of any one of paragraphs 1-59, wherein the yield enhancing composition comprises a beta-glucanase.
61. The method of any one of paragraphs 1-60, wherein the yield enhancing composition comprises a pectinase.
62. The method of any one of paragraphs 1-61, wherein the yield enhancing composition comprises a beta-glucanase and a pectinase.
63. The method of any one of paragraphs 1-62, wherein the yield enhancing composition comprises a cellulase, a xylanase, a protease, and a triacylglycerol lipase.
64. The method of any one of paragraphs 1-63, wherein the at least two, at least three, at least four, or at least five different types of enzymes are selected from the group consisting of an amylase, a beta-glucanases, a cellulase, a hemicellulase, a lipase, a pectinase, and a protease.
65. The method of any one of paragraphs 1-64, wherein the yield enhancing composition comprises at least one fungal strain.
66. The method of any one of paragraphs 1-65, wherein the yield enhancing composition comprises at least one bacterial strain.
67. The method of any one of paragraphs 1-66, wherein the yield enhancing composition comprises at least one fungal strain and at least one bacterial strain.
68. The method of any one of paragraphs 1-67, wherein the yield enhancing composition comprises at least one bacterial strain of the genus Bacillus.
69. The method of any one of paragraphs 1-68, wherein the yield enhancing composition comprises at least one bacterial strain selected from the group consisting of Bacillus amyloliquefaciens, Bacillus megaterium, Bacillus pumilus, Bacillus subtilis, Bacillus thuringiensis, and any combination thereof.
70. The method of any one of paragraphs 1-69, wherein the yield enhancing composition comprises Bacillus amyloliquefaciens, Bacillus megaterium, Bacillus pumilus, Bacillus subtilis, and Bacillus thuringiensis.
71. The method of any one of paragraphs 1-70, wherein the yield enhancing composition comprises at least one bacterial strain of the genus Pseudomonas.
72. The method of any one of paragraphs 1-71, wherein the yield enhancing composition comprises Pseudomonas monteilii.
73. The method of any one of paragraphs 1-72, wherein the yield enhancing composition comprises bacterial strains of the genera Bacillus and Pseudomonas.
74. The method of any one of paragraphs 1-73, wherein the yield enhancing composition comprises Bacillus amyloliquefaciens, Bacillus megaterium, Bacillus pumilus, Bacillus subtilis, Bacillus thuringiensis, and Pseudomonas monteilii 75. The method of any one of paragraphs 1-74, wherein the yield enhancing composition comprises a cellulase, a hemicellulase, Bacillus amyloliquefaciens, Bacillus megaterium, Bacillus pumilus, Bacillus subtilis, Bacillus thuringiensis, and Pseudomonas monteilii.
76. The method of any one of paragraphs 1-75, wherein the yield enhancing composition comprises a cellulase, a xylanase, Bacillus amyloliquefaciens, Bacillus megaterium, Bacillus pumilus, Bacillus subtilis, Bacillus thuringiensis, and Pseudomonas monteilii.
77. The method of any one of paragraphs 1-76, wherein the yield enhancing composition comprises a cellulase, a xylanase, a protease, a lipase, Bacillus amyloliquefaciens, Bacillus megaterium, Bacillus pumilus, Bacillus subtilis, Bacillus thuringiensis, and Pseudomonas monteilii.
78. The method of any one of paragraphs 1-77, wherein the yield enhancing composition comprises a cellulase, a xylanase, a protease, a triacylglycerol lipase, Bacillus amyloliquefaciens, Bacillus megaterium, Bacillus pumilus, Bacillus subtilis, Bacillus thuringiensis, and Pseudomonas monteilii.
79. The method of any one of paragraphs 1-78, wherein the yield enhancing composition is formulated to increase fermentation product yield (e.g., ethanol yield), biogas yield, the rate of biogas production, and combinations thereof.
80. A method for carrying out the combined operation of a bioethanol production unit and a biogas unit, wherein:
   a) milled grain from a dry milling step is mashed with whole stillage and/or thin stillage and/or outflow from the biogas unit,
   b) the mash from 1a) is fed to a cooking stage with mash temperatures below the gelatinization temperature of the starch in the milled grain, followed by an ethanol-forming fermentation step and then feeding the fermented mash to a distillation step to produce a whole stillage;
   wherein the whole stillage from 1b) is fed to the mashing step in a) and/or to the biogas unit and/or to a solid-liquid separation step to generate thin stillage and wet cake; and/or
   wherein the thin stillage is fed to the mashing step in 1a) and/or to the biogas unit and/or used to make corn oil and residual materials resulting from purification of the corn oil; and/or
   wherein the residual materials resulting from purification of the corn oil are fed to the biogas unit; and/or
   wherein the thin stillage is used to produce a protein product with a raw protein content of more than 44 percent and residual materials resulting from purification of the protein product; and/or
   wherein the residual materials resulting from purification of the protein product are fed to the biogas unit; and/or
   wherein the wet cake is fed to the mashing in step 1a) and/or to the biogas unit; and
   wherein a yield enhancing composition comprising at least one, at least two, at least three, at least four, or at least five different types of enzymes and/or at least one, at least two, at least three, at least four, or at least five different microorganism strains is:
i) added to the biogas unit; and/or
ii) added to the whole stillage from 1b) that is fed to the mashing step in 1a); and/or
iii) added to the whole stillage from 1b) that is fed to the biogas unit; and/or
iv) added to the outflow of the biogas unit before being mashed with the corn meal from step 1a); and/or
v) added to the thin stillage that is fed to the mashing step; and/or
v) added to the residual materials resulting from purification of the corn oil that are fed to the biogas unit; and/or
vi) added to the residual materials resulting from purification of the protein product that are fed to the biogas unit; and/or
vii) added to the wet cake that is fed to the mashing step; and/or
viii) added to the wet cake that is fed to the biogas unit; and/or
ix) added to the biogas unit; and/or
x) biomass is added to any one of i)-ix).
81. The method of paragraph 80, wherein the yield enhancing composition comprises the yield enhancing composition of any one of the preceding paragraphs.

### Materials

Yield Enhancing Composition 1: composition comprising cellulase (e.g., endoglucanase), alpha-amylase, protease, triacylglycerol lipase enzymes and Bacillus amyloliquefaciens, Bacillus megaterium, Bacillus pumilus, Bacillus subtilis, Bacillus thuringiensis, and Pseudomonas monteilii bacterial strains.
Yield Enhancing Composition 2: composition comprising endoglucanase.
Yield Enhancing Composition 3: composition comprising cellobiohydrolase.
Yield Enhancing Composition 4: composition comprising beta-glucosidase.
Yield Enhancing Composition 5: composition comprising beta-glucosidase, cellobiohydrolase, and endoglucanase.
Yield Enhancing Composition 6: composition comprising beta-glucosidase, cellobiohydrolase I, cellobiohydrolase II, AA9 polypeptide, xylanase and beta-xylosidase.
Yield Enhancing Composition 7: composition comprising beta-glucosidase, cellobiohydrolase I, cellobiohydrolase II, AA9 polypeptide, xylanase, beta-xylosidase, and protease.
Yield Enhancing Composition 8: composition comprising beta-glucosidase, cellobiohydrolase, endoglucanase, arabinofuranosidase, xylanase.
Yield Enhancing Composition 9: composition comprising beta-glucosidase, cellobiohydrolase, endoglucanase, arabinofuranosidase, xylanase and protease.
Yield Enhancing Composition 10: composition comprising lipase.
Yield Enhancing Composition 11: composition comprising protease.
Yield Enhancing Composition 12: composition comprising cellulase and xylanase.
Yield Enhancing Composition 13: composition comprising alpha-amylase.
Yield Enhancing Composition 14: composition comprising alpha-amylase and glucoamylase.
Yield Enhancing Composition 15: composition comprising beta-glucanase.
Yield Enhancing Composition 16: composition comprising pectinase.
Yield Enhancing Composition 17: composition comprising beta-glucanase and pectinase.

### Example 1: Thin Stillage

Bio Methane Potential tests (BMPs) are conducted in 500 mL anaerobic reactors utilizing AMPTS II systems from BPC Instruments. The AMPTS II instrument is an analytical device that measures ultra-low bio-methane flows from the anaerobic batch digestion of biodegradable substrates.

For each substrate, 9 different treatments listed in Table 1 are evaluated. Each treatment is run in triplicate in 500 mL anaerobic reactors. Reactors are fed with 300 grams of substrate and seeded with 60 grams of active sludge from an industrial anerobic digester processing cow manure. Enzyme and microbial treatments are added at a rate of 0.1% weight % of treatment product vs. dry mass of substrate.

Gas production is monitored by the AMPTS for 45 days. Gas is recirculated through a handheld gas analyzer weekly to determine the concentration of CO2 in order to calculate the remaining concentration and volume of methane produced. Differences in methane production are evaluated on the basis of methane yield per gram of volatile substrate (mL CH4/g VS).

Total solids (TS) is determined by drying at 105 degree Celsius until no further weight change occurs. Ash is determined in a furnace by heating the sample to 550 degree Celsius in a crucible until no further weight change occurs. Volatile solids (VS) is calculated by subtracting total solids with ash content.

The enzyme and microbial treatments are tested for their ability to provide an improved biogas yield and/or increased rate of biogas production. The list of enzymes and microbial products tested is shown in Table 1.

TABLE 1

| Treatment | Addition rate * |
|---|---|
| Control | N.A. |
| Yield Enhancing Composition 1 | 0.1-0.2% wt. product to wt. TS |
| | substrate |
| Yield Enhancing Composition 2 | 0.1-0.2% wt. product to wt. TS |
| | substrate |
| Yield Enhancing Composition 3 | 0.1-0.2% wt. product to wt. TS |
| | substrate |
| Yield Enhancing Composition 4 | 0.1-0.2% wt. product to wt. TS |
| | substrate |
| Yield Enhancing Composition 5 | 0.1-0.2% wt. product to wt. TS |
| | substrate |
| Yield Enhancing Composition 6 | 0.1-0.2% wt. product to wt. TS |
| | substrate |
| Yield Enhancing Composition 7 | 0.1-0.2% wt. product to wt. TS |
| | substrate |
| Yield Enhancing Composition 8 | 0.1-0.2% wt. product to wt. TS |
| | substrate |
| Yield Enhancing Composition 9 | 0.1-0.2% wt. product to wt. TS |
| | substrate |
| Yield Enhancing Composition 10 | 0.1-0.2% wt. product to wt. TS |
| | substrate |
| Yield Enhancing Composition 11 | 0.1-0.2% wt. product to wt. TS |
| | substrate |
| Yield Enhancing Composition 12 | 0.1-0.2% wt. product to wt. TS |
| | substrate |
| Yield Enhancing Composition 13 | 0.1-0.2% wt. product to wt. TS |
| | substrate |
| Yield Enhancing Composition 14 | 0.1-0.2% wt. product to wt. TS |
| | substrate |
| Yield Enhancing Composition 15 | 0.1-0.2% wt. product to wt. TS |
| | substrate |
| Yield Enhancing Composition 16 | 0.1-0.2% wt. product to wt. TS |
| | substrate |
| Yield Enhancing Composition 17 | 0.1-0.2% wt. product to wt. TS |
| | substrate |

* Addition rates are expressed in % wt of the product to wt. of the total solids (TS) of substrate

Thin stillage from an industrial ethanol plant is obtained. Total solids of the thin stillage is approximately 6% w/w solids. 60 grams of active sludge from an industrial anerobic digester processing cow manure is added to 300 grams of thin stillage. Treatments are added as presented in Table 1. Each treatment is conducted in triplicate in 500 mL anaerobic reactors.

Results to be evaluated: The extent to which the treatment increases methane yield is evaluated on the basis of methane volume per gram of volatile substrate (mL CH4/g VS) on a daily basis from the start of digestion to 45 days. The extent to which the treatment increases the rate of biogas production is increased at 7, 14, 28 or 30 day time points is also evaluated.

### Example 2: Whole Stillage

Whole stillage from a commercial ethanol plant is obtained. Total solids of the whole stillage is approximately 10% w/w solids with approximately 65% volatile solids. 60 grams of active sludge from an industrial anerobic digester processing cow manure is added to 300 grams of whole stillage. Treatments are added as presented in Table 1. Each treatment is conducted in triplicate in 500 mL anaerobic reactors.

Results to be evaluated: The extent to which the treatment increases methane yield is evaluated on the basis of methane volume per gram of volatile substrate (mL CH4/g VS) on a daily basis from the start of digestion to 45 days. The extent to which the treatment increases the rate of biogas production is increased at 7, 14, 28 or 30 day time points is also evaluated.

### Example 3: Defatted Syrup

Defatted syrup from an industrial corn to ethanol plant is obtained. Total solids of the defatted syrup is approximately 35% w/w solids with approximately 60% volatile solids. The defatted syrup is diluted 1:1 with water to create a diluted defatted syrup. 60 grams of active sludge from an industrial anerobic digester processing cow manure is added to 300 grams of diluted defatted syrup. Treatments are added as presented in Table 1. Experiments are conducted in triplicate in 500 mL anaerobic reactors.

Results to be evaluated: The extent to which the treatment increases methane yield is evaluated on the basis of methane volume per gram of volatile substrate (mL CH4/g VS) on a daily basis from the start of digestion to 45 days. The extent to which the treatment increases the rate of biogas production is increased at 7, 14, 28, or 30 day time points is also evaluated.

### Example 4: Thin Stillage Codigested with Pig Manure

Thin stillage from a commercial ethanol plant are obtained. Pig manure is obtained from an industrial pig farm. Thin stillage is combined with pig manure to form a thin stillage and pig manure mixture. Active sludge from an industrial anerobic digester processing cow manure will be added to the thin stillage and pig manure mixture. Treatments are added as presented in Table 1. Experiments are conducted in triplicate in 500 mL anaerobic reactors.

Results to be evaluated: The extent to which the treatment increases methane yield is evaluated on the basis of methane volume per gram of volatile substrate (mL CH4/g VS) on a daily basis from the start of digestion to 45 days. The extent to which the treatment increases the rate of biogas production is increased at 7, 14, 28, or 30 day time points is also evaluated.

### Example 5: Thin Stillage Codigested with Corn Sillage

Thin stillage from a commercial ethanol plant are obtained. Corn Sillage is obtained from an industrial corn farm. Thin stillage will be combined with Corn Sillage to form a thin stillage and corn sillage mixture. Active sludge from an industrial anerobic digester processing cow manure is added to the thin stillage and corn sillage mixture. Treatments are added as presented in Table 1. Experiments are conducted in triplicate in 500 mL anaerobic reactors.

Results to be evaluated: The extent to which the treatment increases methane yield is evaluated on the basis of methane volume per gram of volatile substrate (mL CH4/g VS) on a daily basis from the start of digestion to 45 days. The extent to which the treatment increases the rate of biogas production is increased at 7, 14, 28, or 30 day time points is also evaluated.

### Example 6: Thin Stillage with Codigested Mixed Food Waste

Thin stillage from a commercial ethanol plant will be obtained. Mixed Food Waste will be obtained from an industrial biogas plant. Thin stillage will be combined with Mixed Food Waste to form a thin stillage and Mixed Food Waste mixture. Active sludge from an industrial anerobic digester processing cow manure will be added to the thin stillage and mixed food waste mixture. Treatments are added as presented in Table 1. Experiments are conducted in triplicate in 500 mL anaerobic reactors.

Results to be evaluated: The extent to which the treatment increases methane yield is evaluated on the basis of methane volume per gram of volatile substrate (mL CH4/g VS) on a daily basis from the start of digestion to 45 days. The extent to which the treatment increases the rate of biogas production is increased at 7, 14, 28, or 30 day time points is also evaluated.

### Example 7: Thin Stillage with Codigested Municipal Solid Waste

Thin stillage from a commercial ethanol plant is obtained. Municipal Solid Waste is obtained from a commercial Biogas plant. Thin stillage is combined with Municipal Solid Waste to form a thin stillage and Municipal Solid Waste mixture. Active sludge from an industrial anerobic digester processing cow manure is added to the thin stillage and Municipal Solid Waste mixture. Treatments are added as presented in Table 1. Experiments are conducted in triplicate in 500 mL anaerobic reactors.

Results to be evaluated: The extent to which the treatment increases methane yield is evaluated on the basis of methane volume per gram of volatile substrate (mL CH4/g VS) on a daily basis from the start of digestion to 45 days. The extent to which the treatment increases the rate of biogas production is increased at 7, 14, 28, or 30 day time points is also evaluated.

### Example 8: Thin Stillage with Codigested Cassava Pulp

Thin stillage from an industrial corn to ethanol plant are obtained. Cassava Pulp is obtained from a Cassava Starch Manufacturing plant. Thin stillage is combined with cassava pulp to form a thin stillage and cassava pulp mixture. Active sludge from an industrial anerobic digester processing cow manure is added to the thin stillage and cassava pulp mixture. Treatments are added as presented in Table 1. Experiments are conducted in triplicate in 500 mL anaerobic reactors.

Results to be evaluated: The extent to which the treatment increases methane yield is evaluated on the basis of methane volume per gram of volatile substrate (mL CH4/g VS) on a daily basis from the start of digestion to 45 days. The extent to which the treatment increases the rate of biogas production is increased at 7, 14, 28, or 30 day time points is also evaluated.

### Example 9: Thin Stillage with Codigested Sugar Beet Pulp

Thin stillage from a commercial ethanol plant is obtained. Sugar beet pulp is obtained from a sugar beet refining plant. Thin stillage is combined with sugar beet pulp to form a thin stillage and sugar beet pulp mixture. Active sludge from an industrial anerobic digester processing cow manure is added to the thin stillage and sugar beet pulp mixture. Treatments are added as presented in Table 1. Experiments are conducted in triplicate in 500 mL anaerobic reactors.

Results to be evaluated: The extent to which the treatment increases methane yield is evaluated on the basis of methane volume per gram of volatile substrate (mL CH4/g VS) on a daily basis from the start of digestion to 45 days. The extent to which the treatment increases the rate of biogas production is increased at 7, 14, 28, or 30 day time points is also evaluated.

### Example 10: Thin Stillage with Codigested Grass Clippings

Thin stillage from a commercial ethanol plant is obtained. Grass clipping are obtained from natural grass land. Thin stillage is combined with grass clippings to form a thin stillage and grass clippings mixture. Active sludge from an industrial anerobic digester processing cow manure is added to the thin stillage and grass clippings mixture. Treatments are added as presented in Table 1. Experiments are conducted in triplicate in 500 mL anaerobic reactors.

Results to be evaluated: The extent to which the treatment increases methane yield is evaluated on the basis of methane volume per gram of volatile substrate (mL CH4/g VS) on a daily basis from the start of digestion to 45 days. The extent to which the treatment increases the rate of biogas production is increased at 7, 14, 28, or 30 day time points is also evaluated.

### Example 11: Thin Stillage with Codigested Straw Pellets

Thin stillage from a commercial ethanol plant is obtained. Straw Pellets are obtained from a feed supply store. Thin stillage is combined with straw pellets to form a thin stillage and straw pellet mixture. Active sludge from an industrial anerobic digester processing cow manure will be added to the thin stillage and straw pellet mixture. Treatments are added as presented in Table 1. Experiments are conducted in triplicate in 500 mL anaerobic reactors.

Results to be evaluated: The extent to which the treatment increases methane yield is evaluated on the basis of methane volume per gram of volatile substrate (mL CH4/g VS) on a daily basis from the start of digestion to 45 days. The extent to which the treatment increases the rate of biogas production is increased at 7, 14, 28, or 30 day time points is also evaluated.

### Example 12: Thin Stillage Codigested with Pretreated Corn Stover

Thin stillage and pretreated corn stover are obtained from a commercial ethanol plant. Thin stillage will be combined with pretreated corn stover to form a thin stillage and pretreated corn stover mixture. Active sludge from an industrial anerobic digester processing cow manure is added to the thin stillage and corn sillage mixture. Treatments are added as presented in Table 1. Experiments are conducted in triplicate in 500 mL anaerobic reactors.

Results to be evaluated: The extent to which the treatment increases methane yield is evaluated on the basis of methane volume per gram of volatile substrate (mL CH4/g VS) on a daily basis from the start of digestion to 45 days. The extent to which the treatment increases the rate of biogas production is increased at 7, 14, 28, or 30 day time points is also evaluated.

1. A method for carrying out the combined operation of a bioethanol production unit and a biogas unit, wherein:
a) milled grain from a dry milling step is mashed with whole stillage and/or thin stillage and/or outflow from the biogas unit,
b) the mash from 1a) is fed to a cooking stage with mash temperatures below the gelatinization temperature of the starch in the milled grain, followed by an ethanol-forming fermentation step and then feeding the fermented mash to a distillation step to produce a whole stillage;
wherein the whole stillage from 1b) is fed to the mashing step in 1a) and/or to the biogas unit and/or to a solid-liquid separation step to generate thin stillage and wet cake; and/or
wherein the thin stillage is fed to the mashing step in 1a) and/or to the biogas unit and/or used to make corn oil and residual materials resulting from purification of the corn oil; and/or
wherein the residual materials resulting from purification of the corn oil are fed to the biogas unit; and/or
wherein the thin stillage is used to produce a protein product with a raw protein content of more than 44 percent and residual materials resulting from purification of the protein product; and/or
wherein the residual materials resulting from purification of the protein product are fed to the biogas unit; and/or
wherein the wet cake is fed to the mashing in step 1a) and/or to the biogas unit; and
wherein a yield enhancing composition comprising at least one, at least two, at least three, at least four, or at least five different types of enzymes and/or at least one, at least two, at least three, at least four, or at least five different microorganism strains is:
i) added to the biogas unit; and/or
ii) added to the whole stillage from 1b) that is fed to the mashing step in 1a); and/or
iii) added to the whole stillage from 1b) that is fed to the biogas unit; and/or
iv) added to the outflow of the biogas unit before being mashed with the corn meal from step 1a); and/or
v) added to the thin stillage that is fed to the mashing step; and/or
vi) added to the residual materials resulting from purification of the corn oil that are fed to the biogas unit; and/or
vii) added to the residual materials resulting from purification of the protein product that are fed to the biogas unit; and/or
viii) added to the wet cake that is fed to the mashing step; and/or
ix) added to the wet cake that is fed to the biogas unit; and/or
x) added to the biogas unit; and/or
xi) biomass is added to any one of i)-x).

2. The method as claimed in claim 1, wherein whole stillage from step 1b) is fed to a solid-liquid separation step to generate thin stillage and wet cake.

3. The method as claimed in claim 1, wherein the thin stillage is fed to the mashing step in 1a).

4. The method as claimed in claim 1, wherein the yield enhancing composition comprising at least two, at least three, at least four, or at least five different enzymes and/or at least one, at least two, at least three, at least four or at least five different microorganism strains are added to the thin stillage that is fed to the mashing step in 1a).

5. The method as claimed in claim 1, wherein the thin stillage is used to make corn oil and residual materials resulting from purification of the corn oil.

6. The method as claimed in claim 1, wherein the residual materials resulting from purification of the corn oil are fed to the biogas unit.

7. The method as claimed in claim 1, wherein the yield enhancing composition comprising at least two, at least three, at least four, or at least five different types of enzymes and/or at least one, at least two, at least three, at least four, or at least five different microorganism strains are added to the residual materials resulting from purification of the corn oil that are fed to the biogas unit.

8. The method as claimed in claim 1, wherein the thin stillage is used to produce a protein product with a raw protein content of more than 44 percent and residual materials resulting from purification of the protein product.

9. The method as claimed in claim 1, wherein the residual materials resulting from purification of the protein product are fed to the biogas unit.

10. The method as claimed in claim 1, wherein the yield enhancing composition comprising at least two, at least three, at least four, or at least five different types of enzymes and/or at least one, at least two, at least three, at least four, or at least five different microorganism strains are added to the residual materials resulting from purification of the protein product that are fed to the biogas unit.

11. The method as claimed in claim 1, wherein the wet cake is fed to the mashing in step 1a).

12. The method as claimed in claim 1, wherein the yield enhancing composition comprising at least two, at least three, at least four, or at least five different enzymes and/or at least one, at least two, at least three, at least four or at least five different microorganism strains are added to the wet cake that is fed to the mashing step in 1a).

13. The method as claimed in claim 1, wherein the wet cake is fed to the biogas unit.

14. The method as claimed in claim 1, wherein a yield enhancing composition comprising at least two, at least three, at least four, or at least five different enzymes and/or at least one, at least two, at least three, at least four, or at least five different microorganism strains is added to the wet cake that is fed to the biogas unit.

15. The method as claimed in claim 1, wherein the milled grain comprises corn meal.

16. The method as claimed in claim 1, wherein a biomass other than the milled grain is fed to the biogas unit.

17. The method as claimed in claim 16, wherein the biomass comprises cellulosic material and/or lignocellulosic material and/or hemicellulosic material and/or starch material and/or pectin material and/or lipid material.

18. The method as claimed in claim 1, wherein the at least one, at least two, at least three, at least four, or at least five different types of enzymes are selected from the group consisting of an amylase, a beta-glucanases, a cellulase, a hemicellulase, a lipase, a pectinase, and a protease.

19.-27. (canceled)

28. The method as claimed in claim 1, wherein the yield enhancing composition is formulated to increase fermentation product yield (e.g., ethanol yield), biogas yield, the rate of biogas production, and combinations thereof.

29. The method as claimed in claim 1, further comprising mixing the thin stillage with an additional material obtained from a farm, industrial plant, or natural plant origin to form a mixture, adding the yield enhancing composition to the mixture to form a combined mixture, and feeding the combined mixture to the biogas unit.

30. The method as claimed in claim 29, wherein the additional material is pig manure, corn sillage, food waste, municipal solid waste, cassava pulp, sugar beet pulp, grass clippings, straw pellets, or pretreated corn stover.
